(19) United States
(12) Patent Application Publication
(10) Pub. No.: US 2024/0216539 A1
(43) Pub. Date: Jul. 4, 2024

(54) VECTOR FOR DELIVERING ACTIVE AGENT TO NERVOUS SYSTEM, AND USE THEREOF

(71) Applicants: TOOLGEN INCORPORATED, Seoul (KR); INDUSTRY-ACADEMIC COOPERATION FOUNDATION, YONSEI UNIVERSITY, Seoul (KR)

(72) Inventors: Jae Young Lee, Seoul (KR); Sang Woo Ham, Seoul (KR); Hee Sook Bae, Seoul (KR); Gyu Bon Cho, Seoul (KR); Jae Hyung Jang, Seoul (KR); Yun Ha Kim, Daejeon (KR); Dong Soo Lee, Seoul (KR)

(21) Appl. No.: 18/590,251

(22) Filed: Feb. 28, 2024

Related U.S. Application Data
(63) Continuation of application No. PCT/KR2021/011709, filed on Aug. 31, 2021.

(51) Int. Cl. A61K 48/00; A61K 47/69; C12N 15/86

(52) U.S. Cl. CPC A61K 48/005; A61K 47/6901; C12N 15/86; C12N 2750/14143

(57) ABSTRACT

The present application relates to a vector for delivering an active agent to the nervous system. In one embodiment of the present application, a vector for delivering an active agent in which one or more TTR ligands are conjugated to the surface of an adeno-associated virus is provided.

### CROSS-REFERENCE TO RELATED APPLICATIONS

This application is a continuation application of PCT Application No. PCT/KR2021/011709, filed on 31 Aug. 2021. The entire disclosure of the application identified in this paragraph is incorporated herein by reference.

### FIELD

The present application relates to a vector for delivering an active agent to the nervous system.

### BACKGROUND

In order to treat or diagnose a specific disease, a target site where the specific disease occurs should be treated with an active substance such as a drug. That is, in order to diagnose or treat a specific disease, the active substance should be delivered to the target site, which is a site where the specific disease occurs. Therefore, in order to treat a disease related to the nervous system (hereinafter referred to as a neurological disorder), the active substance needs to be delivered to the nervous system. Here, the nervous system is a concept that includes the central nervous system and the peripheral nervous system.

Among them, in particular, the central nervous system is protected from the variable environment by circulating blood, through the blood-brain barrier (BBB) and the blood-cerebrospinal fluid barrier (BCSFB). Meanwhile, the blood-brain barrier, also referred to as the brain-vascular barrier, is a highly selective semi-permeable boundary that prevents the non-selective passage of solutes from circulating blood into the extracellular fluid of the central nervous system, where neurons are located. The blood-cerebrospinal fluid barrier refers to the barrier between the circulating blood and the space in which the cerebrospinal fluid is present. (Engelhardt, Britta, and Lydia Sorokin. “The blood-brain and the blood-cerebrospinal fluid barriers: function and dysfunction.” Seminars in immunopathology. Vol. 31. No. 4. Springer-Verlag, 2009; and Deisenhammer, Florian, Charlotte E. Teunissen, and Hayrettin Tumani. Cerebrospinal fluid in neurologic disorders. Elsevier, 2017.)

As mentioned above, in order to treat central nervous system diseases, an active substance needs to be delivered to the central nervous system, but the difficulty in delivering the active substance to the central nervous system due to the BBB and BCSFB, which protect the central nervous system, is considered a problem.

According to studies conducted to date, researchers have delivered an active substance to the central nervous system using an adeno-associated virus (AAV) as a vector, which is the carrier of the active substance, and by administering the vector intrathecally to the central nervous system. However, even though the intrathecal route is used, it is still difficult to deliver the active substance to the deep brain, and problems of the intrathecally administered vector leaking from the cerebrospinal fluid into the bloodstream and accumulating in the liver are still reported.

The inventors of the present application invented a vector for successfully delivering an active substance to the central nervous system or the peripheral nervous system and a method capable of successfully delivering an active substance to the central nervous system or the peripheral nervous system, using the characteristics of TTR present in cerebrospinal fluid.

### SUMMARY

### Technical Problem

The present application provides a vector for delivering an active substance to the central nervous system, the peripheral nervous system, or nerve cells.

### Technical Solution

In the present application, a vector, for delivering an active substance, in which one or more TTR ligands are conjugated to the surface of an adeno-associated virus, and a use thereof are provided.

The vector for delivering an active substance according to an exemplary embodiment of the present application, in which a TTR ligand is conjugated to the surface thereof, may bind to transthyretin (TTR) in cerebrospinal fluid to be delivered to the central nervous system, the peripheral nervous system, or nerve cells.

### Advantageous Effects

A vector for delivering an active substance provided by the present application may be delivered to the nervous system when administered into cerebrospinal fluid.

The vector for delivering an active substance provided by the present application may be delivered to the nervous system, peripheral nervous system, or nerve cells when administered into cerebrospinal fluid.

The vector for delivering an active substance provided by the present application does not leak into circulating blood when administered into cerebrospinal fluid.

The vector for delivering an active substance provided by the present application does not accumulate in the liver when administered into cerebrospinal fluid.

That is, when the vector for delivering an active substance provided by the present application is used, the active substance can be successfully delivered to the central nervous system, the peripheral nervous system, or nerve cells.

### BRIEF DESCRIPTION OF DRAWINGS

FIG. 1 is a schematic view of a vector for delivering an active substance according to an embodiment of the present application and a method for preparing the same.

FIG. 2 illustrates the binding site of T4, which is an example of a TTR ligand, to transthyretin (TTR).

FIG. 3 illustrates the H¹ NMR results of N-acetyl thyroxine.

FIG. 4 shows the results of measuring the zeta potential of T4-AAV9 of Sample 2.

FIG. 5 shows the results of measuring the zeta potential of T4-AAV2 of Sample 6.

FIG. 6 shows the results of analyzing the ability to introduce into cells of each of Sample 2, Sample 3, Sample 4, and Sample 5 of T4-AAV9.

FIG. 7 shows the results of analyzing the ability to introduce into cells of each of Sample 2, Sample 3, Sample 4, and Sample 5 of T4-AAV9.

FIG. 8 shows the results of analyzing the ability to introduce into cells of Sample 6 of T4-AAV2.

FIG. 9 shows the results of analyzing the ability to introduce into cells of Sample 7 of T4-AAVr3.45.

FIGS. 10, 11, 12, 13, 14 and 15 show the results of analyzing the fluorescence observed in the brain after administering T4-AAV9 including the Luc gene into the cerebrospinal fluid of mice. Among them, FIGS. 11 to 14 are enlarged views of the results shown in FIG. 10 for each condition.

FIG. 16 shows the results of analyzing the fluorescence observed in the liver after administering T4-AAV9 including the Luc gene into the cerebrospinal fluid of mice.

FIGS. 17, 18, 19, 20, 21 and 22 show the results of analyzing the fluorescence observed in some tissues of the brain after administering T4-AAV9 including the GFP gene into the cerebrospinal fluid of mice. Among them, FIGS. 18 to 21 are enlarged views of the results shown in FIG. 17 for each tissue.

FIG. 23 shows the results of analyzing the fluorescence observed in the striatum of the brain after administering T4-AAV9 including the GFP gene into the cerebrospinal fluid of mice.

FIG. 24 shows the results of analyzing the fluorescence observed in the hippocampus of the brain after administering T4-AAV9 including the GFP gene into the cerebrospinal fluid of mice.

FIGS. 25 and 26 show the results of analyzing the fluorescence observed in the liver after administering T4-AAV9 including the GFP gene into the cerebrospinal fluid of mice.

### MODE OF THE INVENTION

In one embodiment, the present application provides a vector for delivering an active substance represented by any one formula selected from Formula 1 and Formulae 1-2 to 1-5.

In one embodiment, the present application provides a composition for delivering an active substance to a target site. In this case, the composition may include a vector for delivering an active substance of the present application and the active substance. In this case, the target site may be the central nervous system, the peripheral nervous system, or nerve cells. Preferably, the target site may be the central nervous system.

In one embodiment, the present application provides a pharmaceutical composition for treating a target disease. In this case, the composition may include a vector for delivering an active substance of the present application and the active substance. In this case, the target disease may be a neurological disorder. Preferably, the target disease may be a central nervous system disease.

In one embodiment of the present application, a vector for delivering an active agent, represented by the following Formula 1, in which one or more TTR ligands are conjugated to the surface of an adeno-associated virus is provided:

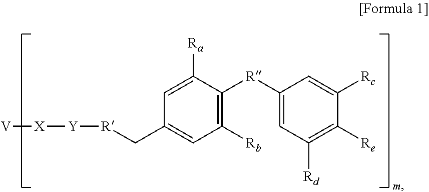

wherein V is an adeno-associated virus, V—X— is an adeno-associated virus moiety, and the adeno-associated virus moiety is derived from an adeno-associated virus,
X is —NH— or —S—, wherein X is derived from a residue of an amino acid present on the capsid of the adeno-associated virus,
Y is -(L)n-, —C(═O)-(L)n-, —C(═NH₂⁺)-(L)n-, —S-(L)n-, or

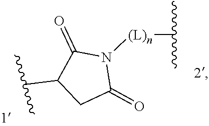

wherein 1′ is linked to X and 2′ is linked to R′,
wherein L is a linker moiety, and L is a substituted or unsubstituted C₁₋₂₀ alkylene, a substituted or unsubstituted C₂₋₂₀ alkenylene, a substituted or unsubstituted C₂₋₂₀ alkynylene, a substituted or unsubstituted C₁₋₂₀ heteroalkylene, a substituted or unsubstituted C₂₋₂₀ heteroalkenylene, or a substituted or unsubstituted C₂₋₂₀ heteroalkynylene,
wherein the heteroalkylene, heteroalkenylene, or heteroalkynylene includes one or more heteroatom groups selected from the group consisting of —NH—, —O—, —S—, —O—N═, —S(═O)—, and —S(═O)₂—, or one or more heteroatoms selected from the group consisting of O, N, and S,
wherein the substitution is substitution with one or more non-hydrogen substituents, and the non-hydrogen substituent is any one selected from the group consisting of —R¹, —C(R¹)₃, —OR¹, —SR¹, —N(R¹)₂, —N═C═O, —NCS, —NO, —NO₂, ═N₂, —N₃, —NHC(═O)R¹, ═O, ═S, —C(═O)R¹, —C(═O)OR¹, —C(═O)NR¹R¹, —S(═O)₂R¹, —OS(═O)₂OR¹, —S(═O)₂NR¹, —S(═O)R¹, alkylene-C(═O)R¹, —C(═S)R¹, alkylene-C(═O)OR¹, —C(═S)OR¹, —C(═O)SR¹, —C(═S)SR¹, —C(═O)NR¹R¹, alkylene-C(═O)NR¹R¹, —C(═S)NR¹R¹, and —C(—NR¹)NR¹R¹, and
wherein R¹ is H, a halogen, a C₁₋₆ alkyl, a C₂₋₆ alkene, a C₂₋₆ alkyne, a C₁₋₆ heteroalkyl, a C₆₋₁₂ aryl, a C₇₋₁₂ arylalkyl or a C₃₋₁₀ cycloalkyl, a C₃₋₁₀ cycloalkene, a C₃₋₁₀ cycloalkyne, or a C₃₋₁₀ heterocycloalkyl,
n is 0 or 1,
R′ is —C(═O)—, a C₁₋₃ alkylene,

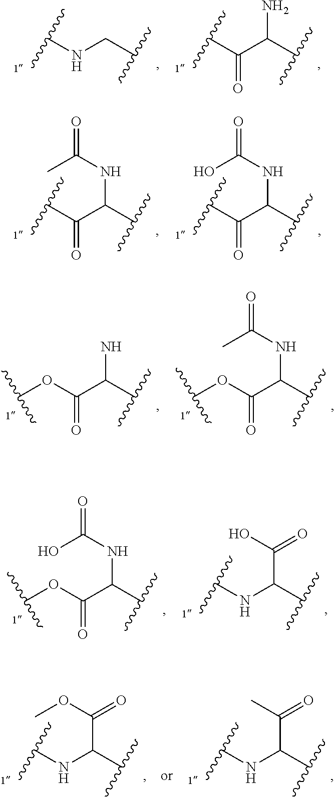

wherein 1″ is linked to Y,
Ra is H, a C₁₋₃ alkyl, or I,
Rb is H, a C₁₋₃ alkyl, or I,
R″ is —O—, —S—, —NH—, or —CH₂—,
Rc is H, a C₁₋₃ alkyl, or I,
Rd is H, a C₁₋₃ alkyl, or I,
Re is OH, and
m is an integer of 2 or greater.

In this case, in the vector for delivering an active substance represented by Formula 1, X may be —NH—, and in this case, X may be derived from the amine residue of lysine located on the capsid of an adeno-associated virus.

In this case, in the vector for delivering an active substance represented by Formula 1, X may be —S—, and in this case, X may be derived from the thiol residue of cysteine located on the capsid of an adeno-associated virus.

In one embodiment of the present application, a vector for delivering an active substance, represented by the following Formula 1-2, in which one or more TTR ligands are conjugated to the surface of an adeno-associated virus is provided:

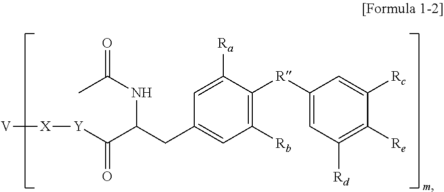

wherein V is an adeno-associated virus, V—X— is an adeno-associated virus moiety, and the adeno-associated virus moiety is derived from an adeno-associated virus,
X is —NH— or —S—, wherein X is derived from a residue of an amino acid present on the capsid of the adeno-associated virus,
Y is -(L)n-, —C(═O)-(L)n-, —C(═NH₂⁺)-(L)n-, —S-(L)n-, or

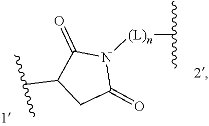

wherein 1′ is linked to X and 2′ is linked to R′,
wherein L is a linker moiety, and L is an unsubstituted C₁₋₁₀ alkylene, an unsubstituted C₂₋₁₀ alkenylene, an unsubstituted C₂₋₁₀ alkynylene, or an unsubstituted C₁₋₁₀ heteroalkylene, and
wherein the heteroalkylene includes one or more heteroatoms selected from the group consisting of O, N, and S,
n is 0 or 1,
Ra is H, a C₁₋₃ alkyl, or I,
Rb is H, a C₁₋₃ alkyl, or I,
R″ is —O—, —S—, —NH—, or —CH₂—,
Rc is H, a C₁₋₃ alkyl, or I,
Rd is H, a C₁₋₃ alkyl, or I,
Re is OH, and
m is an integer of 2 or greater.

In one embodiment of the present application, a vector for delivering an active substance, represented by the following Formula 1-3, in which one or more TTR ligands are conjugated to the surface of an adeno-associated virus is provided:

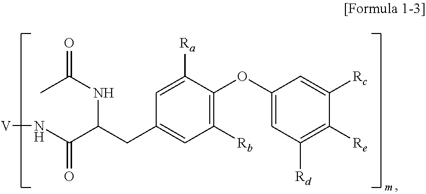

wherein V—NH— is an adeno-associated virus moiety, and wherein the adeno-associated virus moiety is derived from an adeno-associated virus,
wherein in the adeno-associated virus moiety, —NH— is derived from the amine residue of lysine located on the capsid of an adeno-associated virus,
Ra is H, methyl, or I,
Rb is H, methyl, or I,
Rc is H, a C₁₋₃ alkyl, or I,
Rd is H, a C₁₋₃ alkyl, or I,
Re is OH, and
m is an integer of 2 or more.

In one embodiment of the present application, a vector for delivering an active substance, represented by the following Formula 1-4, in which one or more TTR ligands are conjugated to the surface of an adeno-associated virus is provided:

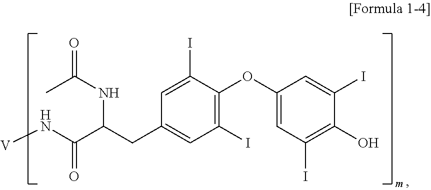

wherein V—NH— is an adeno-associated virus moiety, wherein the adeno-associated virus moiety is derived from an adeno-associated virus,
wherein in the adeno-associated virus moiety, —NH— is derived from the amine residue of lysine located on the capsid of the adeno-associated virus, and
m is an integer of 2 or more.

In one embodiment of the present application, a vector for delivering an active substance, represented by the following Formula 1-5, in which one or more TTR ligands are conjugated to the surface of an adeno-associated virus is provided:

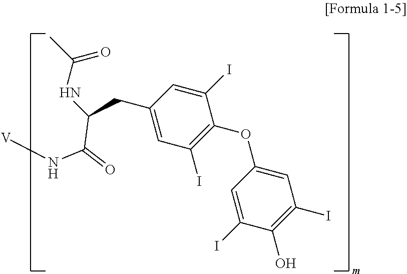

wherein V—NH— is an adeno-associated virus moiety, and wherein the adeno-associated virus moiety is derived from an adeno-associated virus,
wherein, in the adeno-associated virus moiety, —NH— is derived from the amine residue of lysine located on the capsid of an adeno-associated virus, and
m is an integer of 2 or more.

In this case, in an embodiment, the vector for delivering an active substance may further include one or more active substances, and in this case, the active substance may be a transgene.

In this case, in an embodiment, the active substance may be included in the viral particle of the adeno-associated virus moiety.

In this case, in an embodiment, the transgene may be a gene associated with the treatment of neurological disorder.

In this case, in an embodiment, the neurological disorder may be a central nervous system disorder or a peripheral nervous system disorder.

In this case, in an embodiment, the gene associated with the treatment of neurological disorder may be any one selected from MECP2, SCN1A, NF2, SNCA, LRRK2, APP, Tau, Nav1.7, C9rof72, SOD1, DYRK1A, IT15, HTT, HEXA, RA11, PRGN, UBE3A, ABCA4, RP1, PAX6, USH2A, NRP1, SMN1, SMN2, GAA, PMP22, TTR, APOEε4, APOEε3, APOEε2, MAPT, GRN, AADC, GBA1, ASPA, CLN2, GLB1, SGSH, NAGLU, IDS, NPC1, SCA1(ATXN1), SCA3(ATXN3), SCA7(ATXN7), TARDBP(TDP-43), FRDA(FXN), SCN9A, SCN10A, and GAN.

In one embodiment of the present application, a composition for delivering an active substance to the central nervous system or the peripheral nervous system, including a vector for delivering an active substance represented by any one of Formula 1 and Formulae 1-2 to 1-5, is provided.

In one embodiment of the present application, a pharmaceutical composition for treating a neurological disorder, including a transgene and a vector for delivering an active substance represented by any one of Formula 1 and Formulae 1-2 to 1-5, is provided.

In this case, in an embodiment, in the pharmaceutical composition for treating a neurological disorder, the transgene may be included in the vector for delivering an active substance, and in this case, the transgene may be a gene associated with the treatment of neurological disorder.

In this case, in an embodiment, in the pharmaceutical composition for treating a neurological disorder, the neurological disorder may be a central nervous system disorder or a peripheral nervous system disorder.

In one embodiment of the present application, a method for treating a neurological disorder in a subject is provided, wherein the method includes administering a vector for delivering an active substance represented by any one formula selected from Formula 1 and Formulae 1-2 to 1-5 into the cerebrospinal fluid of the subject.

In this case, in an embodiment, the vector may be administered into the cerebrospinal fluid by intrathecal injection or intracerebroventricular injection.

### DETAILED DESCRIPTION

The present application provides a vector for delivering an active substance.

The present application provides a vector for delivering an active substance in which one or more TTR ligands are conjugated to the surface of an adeno-associated virus.

Through the vector for delivering an active substance of the present application, the active substance may be successfully delivered to the nervous system, for example, to the central nervous system or to the peripheral nervous system.

### Definitions

Unless otherwise defined, all technical and scientific terms used in the present application have the same meaning as commonly understood by those of ordinary skill in the art to which the present application pertains.

As used in the present application, the following terms have the meanings conferred to them below, unless otherwise specified.

In some examples, chemical structures are disclosed along with corresponding chemical names. In case of a dispute, the meaning should be grasped by the chemical structure, which takes precedence over the chemical name.

“Halogen” or “halo” refers to a group including fluorine, chlorine, bromine, and iodine, which are included in the halogen group of elements in the periodic table.

As used herein, the term “hetero” refers to a compound or group including at least one heteroatom. The term “heteroatom” refers to an atom other than carbon or hydrogen, and includes, for example, B, Si, N, P, O, S, and Se, and preferably polyvalent elements such as N, O, and S among them, or monovalent elements such as F, Cl, Br, and I, but are not limited thereto.

As used herein, the term “substituted” means that one or more hydrogen atoms on an atom are replaced with a substituent including deuterium and hydrogen variants, where the valence of the atom is normal and the substituted compound is stable. When the substituent is oxygen (that is, ═O), this means that two hydrogen atoms are substituted. When one substituent is a halogen (for example, Cl, F, Br, and I, and the like), this means that one hydrogen atom is substituted with a halogen. When two or more substituents are present in one group, the substituents present in the group may be the same or different. The term “optionally substituted” means that an atom may or may not be substituted with a substituent. Unless otherwise specified, the type and number of substituents may be arbitrary as long as chemically achievable.

An “alkyl” or “alkane” is a straight chain or branched hydrocarbon that is completely saturated. Generally, the alkyl group has 1 to about 20 carbon atoms, preferably 1 to about 10 carbon atoms, unless otherwise defined. The straight chain and branched alkyl groups are, for example, methyl, ethyl, n-propyl, iso-propyl, n-butyl, sec-butyl, tert-butyl, iso-butyl, pentyl, hexyl, heptyl, octyl, nonyl, decyl, and the like. The alkyl group may include a cyclic structure. The term “C_{x-y}” is intended to include residues including x to y carbons in the chain or ring, for example, when used with an alkyl group. For example, the term “C_{x-y} alkyl” refers to an alkyl group which includes x to y carbons in the chain including substituted or unsubstituted form, a straight chain alkyl group, a branched alkyl group, or a cyclic structure and includes, for example, a haloalkyl group such as difluoromethyl and 2,2,2-trifluoroethyl. Co alkyl means hydrogen. Examples of a C₁₋₄ alkyl include methyl, ethyl, n-propyl, iso-propyl, n-butyl, sec-butyl, tert-butyl, iso-butyl, difluoromethyl, 2,2,2-trifluoroethyl, and the like, but are not limited thereto.

As used herein, the term “heteroalkyl” refers to an alkyl including one or more heteroatoms.

An “alkenyl” or “alkene” is a straight chain or branched non-aromatic hydrocarbon including at least one double bond. Typically, the straight chain or branched alkenyl group has 2 to about 20, preferably 2 to about 10 carbon atoms, unless otherwise defined. The alkenyl group may include a cyclic structure.

As used herein, the term “heteroalkene” refers to an alkene including one or more heteroatoms.

An “alkynyl” or “alkyne” is a straight chain or branched non-aromatic hydrocarbon including at least one triple bond. Typically, the straight chain or branched alkynyl group has 2 to about 20, preferably 2 to about 10 carbon atoms, unless otherwise defined. The alkynyl group may include a cyclic structure.

As used herein, the term “heteroalkyne” refers to an alkyne including one or more heteroatoms.

The term “alkylene,” which is used to a molecule or used to a part of a molecule, refers to a divalent radical derived from an alkyl. Examples of an alkylene may include —CH₂—, —CH₂CH₂—, —CH₂CH₂CH₂—, and —CH₂CH₂CH₂CH₂—, but are not limited thereto. For example, alkylene may be written as C₂ alkylene, which refers to an alkylene group including two carbon atoms in the main chain.

The term “heteroalkylene,” which is used to a molecule or used to a part of a molecule, refers to a divalent radical derived from heteroalkyl. Examples of the heteroalkylene group include —CH₂—CH₂—O—CH₂—CH₂— and —CH₂—O—CH₂—CH₂—NH—CH₂—, but are not limited thereto. The heteroalkylene group may include one or more heteroatoms at a position that is not the end of a chain or branch, and each heteroatom may be the same or different. The heteroalkylene group may include one or more heteroatoms at each end of a chain or branch or all the ends of the chain or branch, and each heteroatom may be the same or different.

The term “alkenylene,” which is used to a molecule or used to a part of a molecule, refers to a divalent radical derived from an alkene. Examples of the alkenylene group include —CH═CH—, —CH₂CH═CHCH₂—, and —CH═CH—CH═CH—, but are not limited thereto.

The term “heteroalkenylene,” which is used to a molecule or used to a part of a molecule, refers to a divalent radical derived from a heteroalkene. The heteroalkenylene group may include one or more heteroatoms at a position that is not the end of a chain or branch, and each heteroatom may be the same or different. The heteroalkenylene group may include one or more heteroatoms at each end of a chain or branch or all the ends of the chain or branch, and each heteroatom may be the same or different.

The term “alkynylene,” which is used to a molecule or used to a part of a molecule, refers to a divalent radical derived from an alkyne. For example, the alkynylene group includes —C≡C—, —CH₂C≡CCH₂—, and —C≡C—C≡C—, but is not limited thereto.

The term “heteroalkynylene,” which is used to a molecule or used to a part of a molecule, refers to a divalent radical derived from a heteroalkyne. The heteroalkynylene group may include one or more heteroatoms at a position that is not the end of a chain or branch, and each heteroatom may be the same or different. The heteroalkynylene group may include one or more heteroatoms at each end of a chain or branch or all the ends of the chain or branch, and each heteroatom may be the same or different.

A “cycloalkane” or “cycloalkyl” group is a fully saturated cyclic hydrocarbon. The “cycloalkyl” includes monocyclic and polycyclic rings. Unless otherwise defined, a monocyclic cycloalkyl group generally has 3 to about 10 carbon atoms, and more generally 3 to 8 carbon atoms. A ring other than the first ring of the polycyclic cycloalkyl may be selected from saturated, unsaturated and aromatic ring. The cycloalkyl includes bicyclic molecules in which one, two or three or more atoms are shared between two rings.

The term “cycloalkyne” or “cycloalkynyl” is a cyclic hydrocarbon including at least one triple bond, and is also referred to as a “strained alkyne.” The “cycloalkynyl” includes monocyclic and polycyclic rings. Unless otherwise defined, a monocyclic cycloalkynyl group generally has 3 to about 10 carbon atoms, and more generally 3 to 8 carbon atoms. A ring other than the first ring of the polycyclic cycloalkynyl may be selected from saturated, unsaturated and aromatic ring. The cycloalkynyl includes bicyclic molecules in which one, two or three or more atoms are shared between two rings.

The compound of the present application may have a specific geometric or stereoisomeric form. When compounds are disclosed in the present application without being specified, isomers such as cis and trans isomers, (−)- and (+)-enantiomers, (R)- and (S)-enantiomers, diastereomers, (D)-isomers, (L)-isomers, and racemates of the compounds are included in the scope of the present application. That is, when a formula disclosed in the present application does not have a specific designation associated with isomers (for example, *, [symbol] , [symbol] , [symbol] , and the like), it means that the disclosed formula includes all possible isomers.

The term “conjugated” in the present application means that a specific molecule or specific compound is linked directly or indirectly to a protein or peptide. For example, when protein A (or peptide A) is conjugated with compound B, if the functional group in protein A that binds to compound B is referred to as A′, the following two cases can be exemplified:
i) a case of direct linkage can be exemplified by where A′ is linked to B by a covalent bond (i.e., structural formula represented as A′-B), and
ii) a case of indirect linkage can be exemplified where A′ is linked to B through an intermediary molecule (such as a linker, C) (i.e. structural formula represented as A′-C-B.)

That is, in the present application, ‘A is conjugated to B’ is used in a sense including both a structure represented by formula A′-B and a structure represented by formula A′-C-B.

As used herein, the term “transgene” refers to an exogenous gene that can be introduced into a cell, transcribed into RNA, and selectively translated to and/or expressed as a polynucleotide or protein under appropriate conditions. In one aspect, RNA, an exogenous polynucleotide, and/or an exogenous protein transcribed, translated, and/or expressed from a transgene imparts desired properties to cells into which the transgene has been introduced or induces a desired treatment effect. In another aspect, the transgene may be introduced into cells by a suitable vehicle such as a plasmid, a viral vector, and a nanoparticle. In still another aspect, when appropriate means are available, the transgene may be wholly or partially integrated into the genome of a host cell genome, for example, by a gene editing technique such as a CRISPR/Cas system, a TALEN-based method, and a ZFN-based method. In yet another aspect, the transgene may be transcribed into a molecule that mediates RNA interference (that is, gene silencing), such as miRNA, siRNA, shRNA, and piRNA. In yet another aspect, the transgene may be transcribed into single guide RNA (sgRNA) or double gRNA of crRNA/tracrRNA in the CRISPR/Cas system or expressed as a CRISPR protein such as Cas9, Cas12, or Cas13. In yet another aspect, the transgene may include a target sequence that is targeted by a gene editing technique such as the CRISPR/Cas system.

As used herein, the term “label” refers to a material capable of reaching a target site to diagnose or image a target disease or the condition of a target site. Examples of the label include a radioactive substance, a fluorescent substance, and the like. Radiolabeling is useful for diagnostic imaging and radiotherapy. A representative radioactive substance includes ¹⁸F, ¹¹C, ¹²⁵I, ¹²³I, ¹²⁴I, ¹³¹I, and ^{99m}Tc, but is not limited thereto. The fluorescent substance is used in a sense encompassing a dye or dye reagent used for fluorescent applications. Furthermore, in the present application, the term “fluorescent substance” is used in a sense encompassing a quencher substance. A molecule which can be used as a dye and a dye reagent is widely known in the art. Examples of a representative fluorescent substance include fluorescein isothiocyanate (FITC), phycoerythrin (PE), cyanine (Cy3), cyanine (Cy5), and the like, but are not limited thereto.

As used herein, the term “drug” refers to a molecule that has therapeutic efficacy against any disease. Drugs according to the present application include those known to those of ordinary skill in the art to be effective against any disease. For example, drugs associated with neurological disorder include analgesics, antimigraine agents, CGRP inhibitors, COX-2 inhibitors, narcotic analgesics, nonsteroidal anti-inflammatory drugs, salicylates, anticonvulsants, AMPA receptor antagonists, barbiturate anticonvulsants, benzodiazepine anticonvulsants, carbamate anticonvulsants, carbonic anhydrase inhibitor anticonvulsants, dibenzazepine anticonvulsants, fatty acid derivative anticonvulsants, gamma-aminobutyric acid analogs, gamma-aminobutyric acid reuptake inhibitors, hydantoin anticonvulsants, miscellaneous anticonvulsants, neuronal potassium channel openers, oxazolidinedione anticonvulsants, pyrrolidine anticonvulsants, succinimide anticonvulsants, triazine anticonvulsants, antiemetics, antivertigo agents, 5HT3 receptor antagonists, anticholinergic antiemetics, other antiemetics, NK1 receptor antagonists, phenothiazine antiemetics, antiparkinson agents, anticholinergic antiparkinson agents, dopaminergic antiparkinsonism agents, other antiparkinson agents, anxiolytics, sedatives, hypnotics, barbiturates, benzodiazepines, cholinergic agonists, cholinesterase inhibitors, CNS psychoanaleptics, drugs used for alcohol dependence, general anesthetics, other central nervous system agents, muscle relaxants, neuromuscular blocking agents, skeletal muscle relaxants, VMAT2 inhibitors, and the like, but are not limited thereto.

As another example, drugs with anticancer efficacy include DM1, DM3, DM4, abrin, ricin A, pseudomonas exotoxin, cholera toxin, diphtheria toxin, tumor necrosis factor, α interferon, β interferon, nerve growth factors, platelet derived growth factors, tissue plasminogen activators, cytokines, apoptosis inducing agents, anti-angiogenic agents, lymphokines, taxane, DNA-alkylating agents, anthracycline, tubulysin analogs, duocarmycin analogs, auristatin E, auristatin F, maytansinoids, cytotoxic agents including reactive polyethylene glycol moieties, taxon, cytochalasin B, gramicidin D, ethidium bromide, emetine, mitomycin, etoposide, tenoposide, vincristine, vinblastine, t. colchicine, doxorubicin, daunorubicin, dihydroxy anthracin dione, mitoxantrone, mithramycin, actinomycin D, 1-dehydrotestosterone, glucocorticoid, procaine, tetracaine, lidocaine, propranolol, puromycin, methotrexate, 6-mercaptopurine, 6-thioguanine, cytarabine, 5-fluorouracil dacarbazine, mechlorethamine, thiotepachlorambucil, meiphalan, carmustine, lomustine, cyclophosphamide, busulfan, dibromomannitol, streptozotocin, mitomycin C, cisplatin, dactinomycin, bleomycin, anthramycin, calicheamicin, abiraterone, bendamustine, bortezomib, carboplatin, cabazitaxel, dasatinib, docetaxel, epirubicin, erlotinib, everolimus, gemcitabine, gefitinib, idarubicin, imatinib, hydroxyurea, lapatinib, leuprorelin, melphalan, nedaplatin, nilotinib, oxaliplatin, pazopanib, pemetrexed, picoplatin, romidepsin, satraplatin, sorafenib, vemurafenib, sunitinib, teniposide, triflatin, and vinorelbine, but are not limited thereto.

In particular, in the present specification, in an example, drugs known to be effective against central nervous system disorder may be used.

As used herein, the term “pharmaceutically acceptable salt” refers to a salt that has the efficacy of a parent agent, which is biological or not undesirable (for example, non-toxic or not harmful to its receptors). A suitable salt may include, for example, an acid salt which may be formed by mixing a solution of a parent agent with a solution of a pharmaceutically acceptable acid such as an inorganic acid such as hydrochloric acid, phosphoric acid, and sulfuric acid, and an organic acid such as methanesulfonic acid, p-toluenesulfonic acid, acetic acid, citric acid, maleic acid, succinic acid, oxalic acid, benzoic acid, tartaric acid, fumaric acid, mandelic acid, glucuronic acid, trifluoroacetic acid or benzoic acid. When a drug involves an acidic moiety (for example: —COOH or a phenolic group), a pharmaceutically acceptable salt includes a salt formed with a suitable organic ligand such as an alkali metal salt (for example: a sodium or potassium salt), an alkaline earth metal salt (for example: a calcium or magnesium salt), and a tetravalent ammonium salt. The pharmaceutically acceptable salt may be prepared from an inorganic or organic acid or an inorganic or organic base.

The term “treatment” refers to an approach for obtaining a beneficial or desired clinical outcome. For the purpose of the present invention, the beneficial or desired clinical outcome includes, but not limited to, alleviation of symptoms, reduction in the extent of the disease, stabilization (that is, not worsening) of the disease state, delaying or slowing the rate of disease progression, prevention of disease, or amelioration or palliation and remission (in part or in whole) of the disease state, whether detectable or undetectable. The treatment refers to both therapeutic treatment and prophylactic or method of prophylactic measures.

A “therapeutically effective amount” (or “effective amount”) refers to an amount of an active ingredient, for example, an agent according to the present invention, sufficient to achieve treatment when administered to a subject or patient. Accordingly, what constitutes a therapeutically effective amount of a composition according to the present invention may be readily determined by those of ordinary skill in the art. Of course, the therapeutically effective amount will vary depending on the particular subject and condition being treated, the body weight and age of the subject, the severity of the disease condition, the particular compound selected, the subsequent dosage regimen, the timing of administration, the mode of administration, and the like, all of which can be readily determined by those skilled in the art.

The “subject” or “patient” refers to an animal in need of treatment that can be achieved by the molecules of the present invention. Animals that can be treated according to the present invention include vertebrates, with mammals such as murine, bovine, canine, equine, feline, ovine, porcine, and primate (including humans and non-human primates) animals being particularly preferred examples.

The term “about” refers to an amount, a level, a value, a number, a frequency, a percentage, a dimension, a size, a quantity, a weight, or a length that varies to the degree of 30, 25, 20, 25, 10, 9, 8, 7, 6, 5, 4, 3, 2, or 1% with respect to a reference amount, level, value, number, frequency, percentage, dimension, size, quantity, weight, or length.

### Problems in Related Art and Solution to Problems

### Problems in Related Art

As described above, an active substance should be delivered to the central nervous system or the peripheral nervous system in order to treat a nervous system disease. Vectors are used to deliver the active substance to the central nervous system or the peripheral nervous system, which is the target site.

In the related art, vectors for delivering an active substance were intended to be intrathecally administered to deliver the active substance to the nervous system. However, even though a vector is administered using the intrathecal route of administration, there is a problem in that (i) the administered vector is not delivered to the central nervous system, the peripheral nervous system, or nerve cells, and there is a problem in that (ii) the administered vector leaks from the cerebrospinal fluid into the blood, and the vector that leaks into the blood accumulates in the liver, resulting in an insufficient amount of vector being delivered to a target site (for example, the deep brain or sciatic nerve) or inability of the vector to reach the target site.

That is, there is a problem in that an existing vector intrathecally administered is not successfully delivered to the central nervous system, the peripheral nervous system, or nerve cells.

In order to solve the above problems, the present application provides a vector for delivering an active substance of the present application, which is described below and has high delivery efficiency to the nervous system.

### Principle of Solving Problem Using Vector for Delivering Active Substance of Present Application

The present application solves the above problems through a vector for delivering an active substance in which one or more transthyretin (TTR) ligands are conjugated to the surface of a virus. That is, the binding specificity of the transthyretin ligand to a specific target is used.

### (1) Transthyretin and Thyroxine

Transthyretin (TTR) is a transport protein present in serum and cerebrospinal fluid, and transports thyroxine (T4) which is a thyroid hormone, and retinol. In the circulating blood, in addition to TTR, albumin, thyroxine-binding globulin (TBG), and the like act as transport proteins for T4, but in cerebrospinal fluid, TTR produced in the choroid plexus is known as the only transport protein for T4. T4 is known to play an important role in the metabolism of nerve cells, and TTR in cerebrospinal fluid does not leak into the circulating blood while transporting T4 to nerve cells. The present application uses this specific interaction between TTR and T4 in cerebrospinal fluid.

### (2) Solving Problems Using Vector for Delivering Active Substance of Present Application

The vector of the present application is characterized in that one or more transthyretin ligands (TTR ligands) are conjugated to the surface of the vector.

In one example, the present application provides a vector for delivering an active substance in which one or more TTR ligands are conjugated to the surface of an adeno-associated virus.

Therefore, when the vector for delivering an active substance of the present application is administered into cerebrospinal fluid, TTR present in the cerebrospinal fluid and a TTR ligand present on the surface of the vector bind. In this case, examples of methods for administering the vector into cerebrospinal fluid include intrathecal injection (IT injection), intracerebroventricular injection (ICV injection), and the like.

(i) As described above, since TTR in cerebrospinal fluid transports T4 (an example of a TTR ligand) to nerve cells, that is, the vector for delivering an active substance of the present application intrathecally administered is bound to TTR present in cerebrospinal fluid and delivered to the nervous system according to the transport route of TTR. This allows a transgene contained in the vector of the present application to be delivered to the nervous system.

(ii) As described above, since TTR present in cerebrospinal fluid does not leak into circulating blood, the vector for delivering an active substance of the present application administered intrathecally is bound to TTR in cerebrospinal fluid, and thus does not leak into circulating blood according to the characteristics of TTR present in cerebrospinal fluid. Therefore, the vector for delivering an active substance of the present application, which does not leak into circulating blood, accumulates in the liver in a small amount. That is, the vector of the present application is present longer in cerebrospinal fluid, so that the probability that the vectors will be delivered to the nervous system, which is the target site, will be higher.

As described above, the vector for delivering an active substance of the present application may be successfully delivered to the central nervous system, the peripheral nervous system, or nerve cells when administered into cerebrospinal fluid. Furthermore, the vector for delivering an active substance of the present application may be successfully delivered to the deep brain or sciatic nerve.

Hereinafter, the vector for delivering an active substance of the present application will be specifically described.

### Vector for Delivering Active Substance of Present Application

The present application provides a vector for delivering an active substance in which one or more TTR ligands are conjugated to the surface of an adeno-associated virus. In a specific example of the vector for delivering an active substance of the present application, a TTR ligand is conjugated to the surface of an adeno-associated virus (AAV). The TTR ligand is conjugated to the surface of an adeno-associated virus through other sites rather than the TTR binding site. Therefore, even though a TTR ligand is conjugated to the surface of an adeno-associated virus, the binding activity of the conjugated TTR ligand to TTR is not reduced.

In an embodiment, the present application provides a vector for delivering an active substance in which one or more TTR ligands are conjugated to the surface of an adeno-associated virus, represented by the following Formula 1:

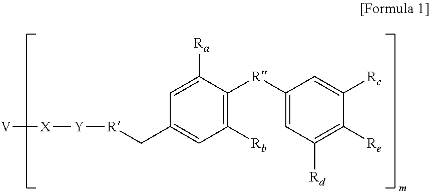

wherein V is an adeno-associated virus, V—X— is named an adeno-associated virus moiety, and the adeno-associated virus moiety is derived from an adeno-associated virus.

In this case, X is —NH— or —S—, and in this case, X is derived from a residue of an amino acid located on the capsid of an adeno-associated virus,
Y is -(L)n-, —C(═O)-(L)n-, —C(═NH₂⁺)-(L)n-, —S-(L)n-, or

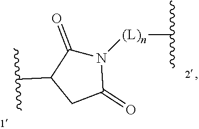

wherein 1′ is linked to X and 2′ is linked to R′,
wherein L is a linker moiety, and L is a substituted or unsubstituted C₁₋₂₀ alkylene, a substituted or unsubstituted C₂₋₂₀ alkenylene, a substituted or unsubstituted C₂₋₂₀ alkynylene, a substituted or unsubstituted C₁₋₂₀ heteroalkylene, a substituted or unsubstituted C₂₋₂₀ heteroalkenylene, or a substituted or unsubstituted C₂₋₂₀ heteroalkynylene,
wherein the heteroalkylene, heteroalkenylene, or heteroalkynylene includes one or more heteroatom groups selected from the group consisting of —NH—, —O—, —S—, —O—N═, —S(═O)—, and —S(═O)₂—, or one or more heteroatoms selected from the group consisting of O, N, and S,
wherein the substitution is substitution with one or more non-hydrogen substituents, and the non-hydrogen substituent is any one selected from the group consisting of —R¹, —C(R¹)₃, —OR¹, —SR¹, —N(R¹)₂, —N═C═O, —NCS, —NO, —NO₂, ═N₂, —N₃, —NHC(═O)R¹, ═O, ═S, —C(═O)R¹, —C(═O)OR¹, —C(═O)NR¹R¹, —S(═O)₂R¹, —OS(═O)₂OR¹, —S(═O)₂NR¹, —S(═O)R¹, alkylene-C(═O)R¹, —C(═S)R¹, alkylene-C(═O)OR¹, —C(═S)OR¹, —C(═O)SR¹, —C(═S)SR¹, —C(═O)NR¹R¹, alkylene-C(═O)NR¹R¹, —C(═S)NR¹R¹, and —C(—NR¹)NR¹R¹,
wherein R¹ is H, a halogen, a C₁₋₆ alkyl, a C₂₋₆ alkene, a C₂₋₆ alkyne, a C₁₋₆ heteroalkyl, a C₆₋₁₂ aryl, a C₇₋₁₂ arylalkyl or a C₃₋₁₀ cycloalkyl, a C₃₋₁₀ cycloalkene, a C₃₋₁₀ cycloalkyne, or a C₃₋₁₀ heterocycloalkyl,
wherein n is 0 or 1,
R′ is —C(═O)—, a C₁₋₃ alkylene,

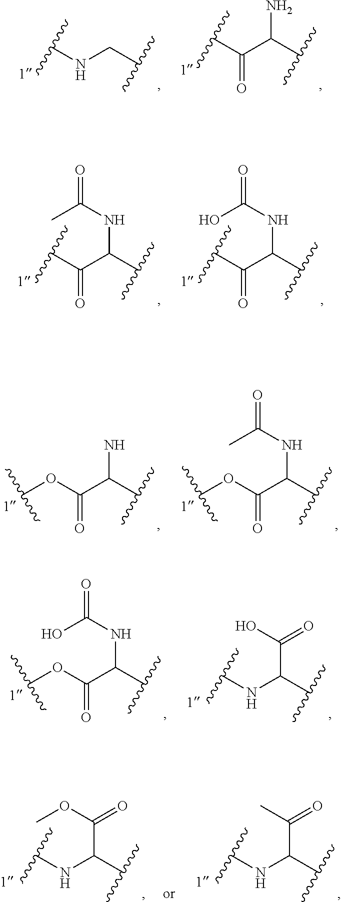

wherein 1″ is linked to Y,
Ra is H, a C₁₋₃ alkyl, or I,
Rb is H, a C₁₋₃ alkyl, or I,
R″ is —O—, —S—, —NH—, or —CH₂—,
Rc is H, a C₁₋₃ alkyl, or I,
Rd is H, a C₁₋₃ alkyl, or I,
Re is OH, and
m is an integer of 2 or more.

In an embodiment, in this case, L may be a substituted or unsubstituted C₁₋₁₀ alkylene, a substituted or unsubstituted C₂₋₁₀ alkenylene, a substituted or unsubstituted C₂₋₁₀ alkynylene, a substituted or unsubstituted C₁₋₁₀ heteroalkylene, a substituted or unsubstituted C₂₋₁₀ heteroalkenylene, or a substituted or unsubstituted C₂₋₁₀ heteroalkynylene.

In an embodiment, in this case, L may be a substituted or unsubstituted C₁₋₅ alkylene, a substituted or unsubstituted C₂₋₅ alkenylene, a substituted or unsubstituted C₂₋₅ alkynylene, a substituted or unsubstituted C₁₋₅ heteroalkylene, a substituted or unsubstituted C₂₋₅ heteroalkenylene, or a substituted or unsubstituted C₂₋₅ heteroalkynylene.

In an embodiment, in this case, the residue of an amino acid located on the capsid of the adeno-associated virus may be an amine residue of lysine or a thiol residue of cysteine.

In an embodiment, in this case, the residue of an amino acid located on the capsid of the adeno-associated virus may be an amine residue of lysine.

In this case, in Formula 1, a moiety represented by the following Formula 3-1 is named an adeno-associated virus moiety, and this is represented by the symbol V. In this case, the adeno-associated virus moiety is derived from an adeno-associated virus.

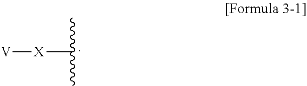

In this case, in Formula 1, a moiety represented by the following Formula 3-2 is named a TTR ligand moiety, and this is represented by the symbol TTRL. In this case, the TTR ligand moiety is derived from a TTR ligand.

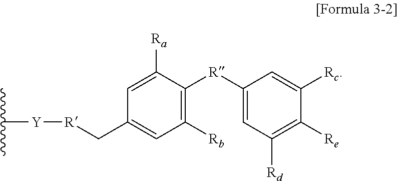

In this case, the description on each symbol in Formulae 3-1 and 3-2 is as described above.

Therefore, the vector for delivering an active substance of the present application may be represented by the symbol V′-TTRL.

The vector for delivering an active substance of the present application is produced by conjugating a TTR ligand to the surface of an adeno-associated virus. In a specific example, the TTR ligand may be conjugated to an amine residue of an amino acid present on the capsid of an adeno-associated virus.

Hereinafter, adeno-associated viruses and TTR ligands will be described.

### Adeno-Associated Virus (AAV)

Adeno-associated viruses are viruses that infect humans and some other primate species, and are currently known to cause no specific disease and to cause a very weak immune response. That is, adeno-associated viruses have non-pathogenic characteristics.

Adeno-associated viruses consist of viral particles and a genetic material included in the viral particles. In this case, the genetic material included in the viral particles is single-stranded DNA, and the surface of the viral particle is composed of a capsid protein. Typically, the transgene is introduced into the viral particles of an adeno-associated virus, and the adeno-associated virus vector is used as a vector for delivering a transgene to any cells.

Examples of adeno-associated viruses which may be used in the present application include AAV1, AAV2, AAV3, AAV4, AAV5, AAV6, AAV7, AAV8, AAV9, AAV10, AAV11, AAV12, AAVr3.45, AAVrh10, AAV106.1/hu.37, AAV114.3/hu.40, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2i8, AAV2i8G9, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV2.5T, AAV27.3, AAV29.3/bb.1, AAV29.5/bb.2, AAV2G9, AAV3B, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rh.64, AAV4-9/rh.54, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6.1, AAV6.1.2, AAV6.2, AAV7m8, AAV7.2, AAV7.3/hu.7, AAV-8b, AAV8G9, AAV-8h, AAV9i1, AAV9.11, AAV9.13, AAV9.16, AAV9.24, AAV9.45, AAV9.47, AAV9.61, AAV9.68, AAV9.84, AAV9.9, AAVcy.2, AAVcy.3, AAVcy.4, AAVcy.5, AAVcy.5R¹, AAVcy.5R², AAVcy.5R³, AAVcy.5R⁴, AAVcy.6, AAVhu.1, AAVhu.2, AAVhu.3, AAVhu.4, AAVhu.5, AAVhu.6, AAVhu.7, AAVhu.8, AAVhu.9, AAVhu.10, AAVhu.11, AAVhu.12, AAVhu.13, AAVhu.14/9, AAVhu.15, AAVhu.16, AAVhu.17, AAVhu.18, AAVhu.19, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R¹, AAVhu.48R², AAVhu.48R³, AAVhu.49, AAVhu.51, AAVhu.52, AAVhu.53, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVpi.1, AAVpi.2, AAVpi.3, AAVrh.2, AAVrh.2R, AAVrh.8, AAVrh.8R, AAVrh8R R533A mutants, AAVrh8R A586R mutants, AAVrh.10, AAVrh.12, AAVrh.13, AAVrh. 13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R¹, AAVrh.64R², AAVrh.65, AAVrh.67, AAVrh.68, AAVrh.69, AAVrh.70, AAVrh.72, AAVrh.73, AAVrh.74, AAV-PHP.B, AAV-PHP.A, AAV-G2B-26, AAV-G2B-13, AAV-TH1.1-32, AAV-TH1.1-35, AAV-PHP.B2, AAV-PHP.B3, AAV-PHP.N/PHP.B-DGT, AAV-PHP.B-EST, AAV-PHP.B-GGT, AAV-PHP.B-ATP, AAV-PHP.B-ATT-T, AAV-PHP.B-DGT-T, AAV-PHP.B-GGT-T, AAV-PHP.B-SGS, AAV-PHP.B-AQP, AAV-PHP.B-QQP, AAV-PHP.B-SNP(3), AAV-PHP.B-SNP, AAV-PHP.B-QGT, AAV-PHP.B-NQT, AAV-PHP.B-EGS, AAV-PHP.B-SGN, AAV-PHP.B-EGT, AAV-PHP.B-DST, AAV-PHP.B-DST, AAV-PHP.B-STP, AAV-PHP.B-PQP, AAV-PHP.B-SQP, AAV-PHP.B-QLP, AAV-PHP.B-TMP, AAV-PHP.B-TTP, AAV-PHP.S/G2A12, AAV-G2A15/G2A3, AAV-G2B4, AAV-G2B5, PHP.S, AAAV, AAVA3.3, AAV A3.4, AAV A3.5, AAV A3.7, AAV CBr-7.3, AAV CBr-7.1, AAV CBr-7.10, AAV CBr-7.2, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-N4, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-M9, AAV CLv-R6, AAV CLv-1, AAV CLv1-1, AAV CLv1-10, AAV CLv1-2, AAV CLv-12, AAV CLv1-3, AAV CLv-13, AAV CLv1-4, AAV CLv1-7, AAV CLv1-8, AAV CLv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-R1, AAV CLv-R2, AAV CLv-R3, AAV CLv-R4, AAV CLv-R5, AAV CLv-R7, AAV CLv-R8, AAV CLv-R9, AAV CSp-8.10, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV-LK08, AAV-LK15, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV.VR-355, AAV-b, AAVC1, AAVC2, AAVC5, AAVCh.5, AAVCh.5R1, AAV-DJ, AAV-DJ8, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3, AAVF3/HSC3, AAVF4/HSC4, AAVF5, AAVF5/HSC5, AAVF6/HSC6, AAVF7/HSC7, AAVF8/HSC8, AAVF9/HSC9, AAV-h, AAVH-1/hu.1, AAVH2, AAVH-5/hu.3, AAVH6, AAVhE1.1, AAVhEr1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVLG-10/rh.40, AAVLG-4/rh.38, AAVLG-9/hu.39, AAVLG-9/hu.39, AAV-LK01, AAV-LK02, AAV-LK03, AAV-LK03, AAV-LK04, AAV-LK05, AAV-LK06, AAV-LK07, AAV-LK09, AAV-LK10, AAV-LK11, AAV-LK12, AAV-LK13, AAV-LK14, AAV-LK16, AAV-LK17, AAV-LK18, AAV-LK19, AAVN721-8/rh.43, AAV-PAEC, AAV-PAEC 12, AAV-PAEC11, AAV-PAEC2, AAV-PAEC4, AAV-PAEC6, AAV-PAEC7, AAV-PAEC8, Anc80, Anc80L65, Anc81, Anc82, Anc83, Anc84, Anc94, Anc110, Anc113, Anc126, Anc127, BAAV, BNP61 AAV, BNP62 AAV, BNP63 AAV, bovine AAV, caprine AAV, Japanese AAV 10 serotype, UPENN AAV10, VOY101, VOY201, or the like.

Preferably, the adeno-associated virus used in the present application may be any one selected from the group consisting of AAV1, AAV2, AAV3, AAV4, AAV5, AAV6, AAV7, AAV8, AAV9, AAV10, AAV11, AAV12, and AAVr3.45.

The case where an adeno-associated virus is used to deliver an active substance has various advantages compared to the case where nanoparticles are used.

When nanoparticles are used, an additional processing is required to design a moiety by which the surface of the nanoparticles can bind to a TTR ligand in order to conjugate the TTR ligand. For example, the surface of the nanoparticles may need to be designed with an amine group or, in the case of lipid nanoparticles, the lipid nanoparticles may need a transmembrane protein capable of binding the TTR ligand, in the phospholipid bilayer of the surface of the lipid nanoparticles. In contrast, when the adeno-associated virus is used, the TTR ligand may be conjugated through a residue of an amino acid present on the capsid without any additional surface treatment.

In many cases, the surface characteristics of adeno-associated viruses do not change even though a carrier material (for example, an active substance) is changed, but, in the case of nanoparticles, there is a disadvantage in that particle properties such as zeta potential or the size of nanoparticles may be changed depending on the carrier material.

In addition, adeno-associated viruses have an advantage of being less cytotoxic than nanoparticles, and have an advantage of having various forms (serotypes) that can also be used clinically in living organisms.

Furthermore, when the adeno-associated virus is used, the transgene delivered into cells through the adeno-associated virus may be present in the form of an epigenome, and thus, may remain for a longer period of time, but when the transgene is delivered using nanoparticles, the transgene may disappear quickly in cells.

Further, adeno-associated viruses have an advantage in that they are better than nanoparticles in terms of the ability to deliver the transgene into the central nervous system or the peripheral nervous system.

### TTR Ligand

The vector of the present application is characterized in that one or more TTR ligands are conjugated to the surface of the AAV described above.

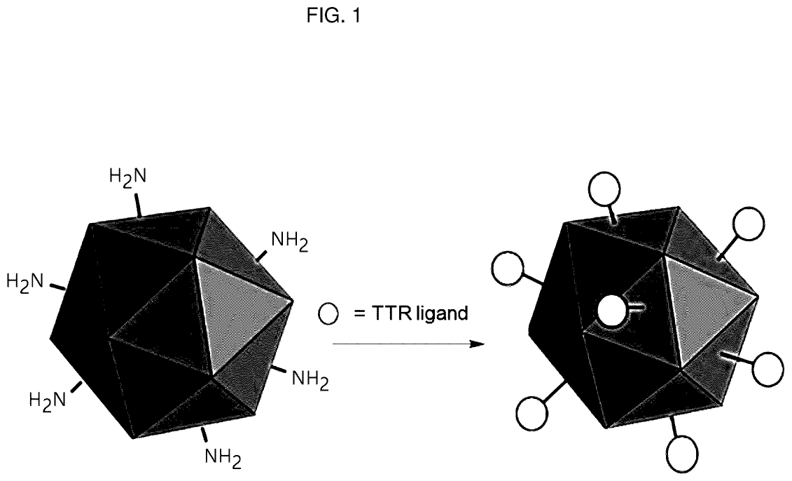

A schematic view for a vector for delivering an active substance provided by an embodiment of the present application and its production are shown in FIG. 1. In an embodiment, the vector for delivering an active substance of the present application includes a TTR ligand moiety (TTRL). In this case, the TTR ligand moiety is derived from the TTR ligand and may be represented by the structure of Formula 3-2 described above.

As used herein, the term ‘TTR ligand’ refers to a compound that has the ability to specifically bind to TTR. The TTR ligand includes commonly known compounds having the ability to bind to TTR and derivatives thereof. In this case, the derivative of the compound having the ability to bind to TTR may be one in which a part of the chemical structure of the compound is modified such that the compound having the ability to bind to TTR can chemically react with other materials.

In one embodiment of the present application, the TTR ligand is represented by the following formula:

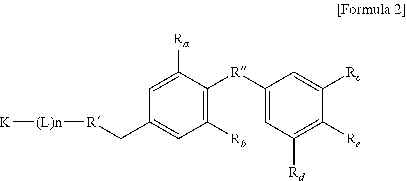

wherein J may be any one selected from R², an N-succinimide group, an N-hydroxysuccinimide group (NHS group), an N-hydroxysuccinimide ester group (NHS ester group), a sulfo-N-hydroxysuccinimide group (sulfo-NHS group), a sulfo-N-Hydroxysuccinimide ester group (sulfo-NHS ester group), an imidoester group, a maleimide group, an iodoacetyl group, a pyridyldithiol group, a p-nitrophenyl group, and a pentafluorophenyl group, and
wherein R² is any one selected from H, OH, NH₂, C₁₋₃ alkyl, —C(═O)OH, —C(═O)CH₃, —C(═O)C₂H₅, and —C(═O)C₃H₇.
L is a linker moiety, and L is substituted or unsubstituted C₁₋₂₀ alkylene, substituted or unsubstituted C₂₋₂₀ alkenylene, substituted or unsubstituted C₂₋₂₀ alkynylene, or substituted or unsubstituted C₁₋₂₀ heteroalkylene,
wherein the heteroalkylene includes one or more heteroatoms or heteroatom groups selected from the group consisting of —NH—, —O—, —S—, —O—N═, —S(═O)—, —S(═O)₂—, and N,
wherein the substitution refers to being substituted with one or more non-hydrogen substituents, and the non-hydrogen substituent is any one selected from the group consisting of —R¹, —C(R¹)₃, —OR¹, —SR¹, —N(R¹)₂, —N═C═O, —NCS, —NO, —NO₂, ═N₂, —N₃, —NHC(═O)R¹, ═O, ═S, —C(═O)R¹, —C(═O)OR¹, —C(═O)NR¹R¹, —S(═O)₂R¹, —OS(═O)₂OR¹, —S(═O)₂NR¹, —S(═O)R¹, alkylene-C(═O)R¹, —C(═S)R¹, alkylene-C(═O)OR¹, —C(═S)OR¹, —C(═O)SR¹, —C(═S)SR¹, —C(═O)NR¹R¹, alkylene-C(═O)NR¹R¹, —C(═S)NR¹R¹, and —C(—NR¹)NR¹R¹,
wherein R¹ is H, halogen, C₁₋₆ alkyl, C₂₋₆ alkene, C₂₋₆ alkyne, C₁₋₆ heteroalkyl, C₆₋₁₂ aryl, C₇₋₁₂ arylalkyl or C₃₋₁₀ cycloalkyl, C₃₋₁₀ cycloalkene, C₃₋₁₀ cycloalkyne, or C₃₋₁₀ heterocycloalkyl,
wherein n is 0 or 1,
R′ is —C(═O)—, a C₁₋₃ alkylene,

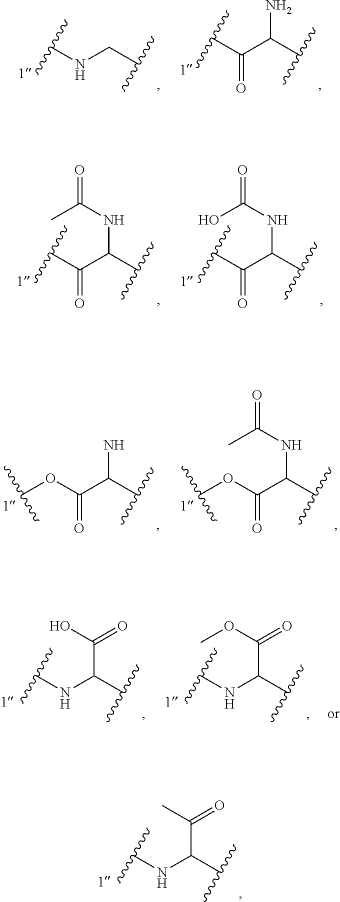

wherein when 1″ is linked to L when n is 1, or is linked to J when n is 0,
Ra is H, C₁₋₃ alkyl, or I,
Rb is H, C₁₋₃ alkyl, or I,
R″ is —O—, —S—, —NH—, or —CH₂—,
Rc is H, C₁₋₃ alkyl, or I,
Rd is H, C₁₋₃ alkyl, or I, and
Re is OH.

In an embodiment, in this case, L may be substituted or unsubstituted C₁₋₁₀ alkylene, substituted or unsubstituted C₂₋₁₀ alkenylene, substituted or unsubstituted C₂₋₁₀ alkynylene, substituted or unsubstituted C₁₋₁₀ heteroalkylene, substituted or unsubstituted C₂₋₁₀ heteroalkenylene, or substituted or unsubstituted C₂₋₁₀ heteroalkynylene.

In an embodiment, in this case, L may be substituted or unsubstituted C₁₋₅ alkylene, substituted or unsubstituted C₂₋₅ alkenylene, substituted or unsubstituted C₂₋₅ alkynylene, substituted or unsubstituted C₁₋₅ heteroalkylene, substituted or unsubstituted C₂₋₅ heteroalkenylene, or substituted or unsubstituted C₂₋₅ heteroalkynylene.

In this case, in the present application, J-(L)n-R′— of Formula 2 refers to a conjugating part, and means a part of the TTR ligand that may conjugate to an adeno-associated virus, and
a structure represented by

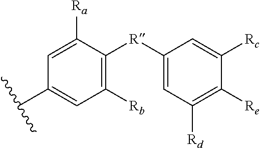

refers to a TTR binding part, and is a part of the TTR ligand that binds to TTR.

Hereinafter, the TTR binding part will be described.

A specific ligand compound has binding specificity to a specific protein. In this case, the ligand compound should be designed such that the ability to bind to the corresponding protein is not reduced.

The TTR ligand of the present application is also designed so as to be able to conjugate to the capsid protein of an adeno-associated virus while maintaining the ability of the TTR ligand to bind to TTR. Therefore, locations other than the TTR binding part are conjugated to the capsid protein of an adeno-associated virus.

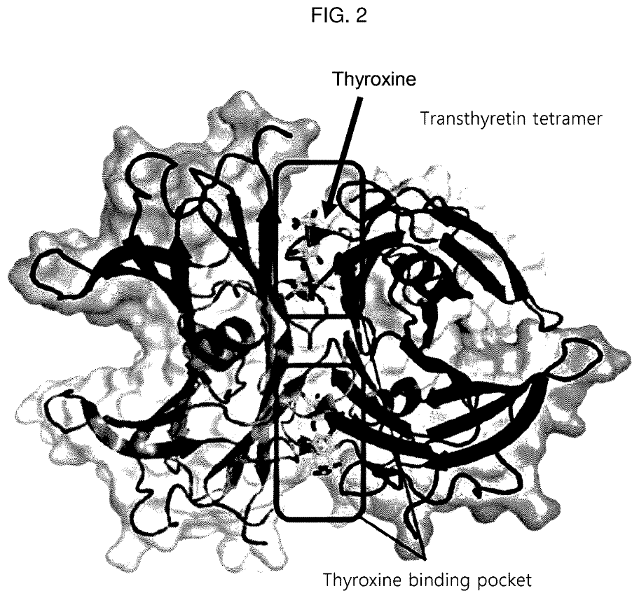

As shown in FIG. 2, when the TTR ligand and TTR interact (for example, bind), the diphenyl group of the TTR ligand is located inside the TTR. In the present application, the diphenyl group of such TTR ligand is referred to as the TTR binding part, and a part other than this part is conjugated to the surface of the adeno-associated virus (the conjugating part is represented by (J-(L)n-R′—)).

Therefore, according to such a binding structure, the ability to bind to TTR, of the TTR ligand conjugated to the adenovirus of the present application may be maintained.

In addition, since the conjugating part of the TTR ligand is located outside of TTR in the interaction between the TTR ligand and TTR, it is more advantageous for the TTR ligand to be conjugated to the adeno-associated virus through the TTR conjugating part rather than the TTR binding part to maintain the ability of the adeno-associated virus-conjugated TTR ligand to bind to TTR.

In one embodiment, the TTR ligand may be any one selected from thyroxine (T4), thyroxine derivatives (T4 derivatives), triiodothyronine (T3), T3 derivatives, reverse T3 (rT3), rT3 derivatives, tetram, tetram derivatives, tetrac, 3,5-dimethyl-4-(4′hydroxy-3′-isopropylbenzyl)-phenoxy acetic acid (GC-1) and derivatives thereof, 3,5-diiodothyropropionic acid (DITPA) and derivatives thereof, and tetrac derivatives, but is not limited thereto.

In one embodiment, the TTR ligand may be any one selected from compounds represented by the following formulae, but is not limited thereto:

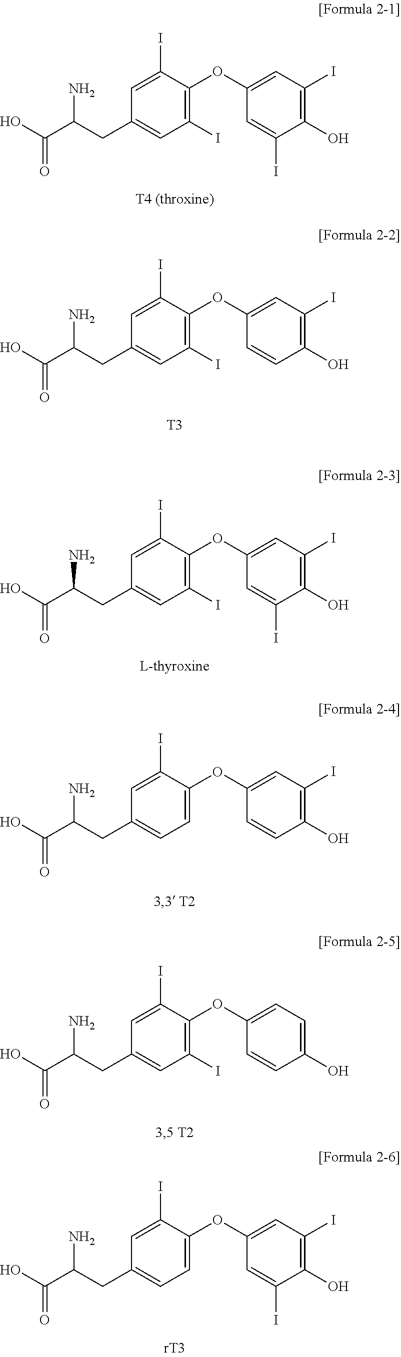

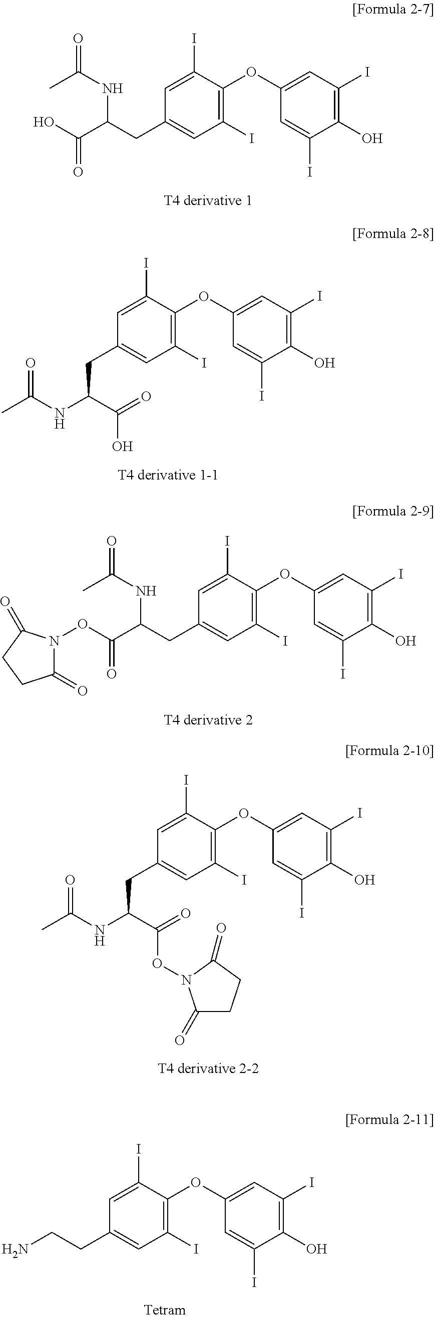 [SMILES: CC(=O)NC(Cc1cc(I)c(Oc2cc(I)c(O)c(I)c2)c(I)c1)C(=O)O ; CC(=O)N[C@@H](Cc1cc(I)c(Oc2cc(I)c(O)c(I)c2)c(I)c1)C(=O)O ; CC(=O)NC(Cc1cc(I)c(Oc2cc(I)c(O)c(I)c2)c(I)c1)C(=O)ON1C(=O)CCC1=O ; CC(=O)N[C@@H](Cc1cc(I)c(Oc2cc(I)c(O)c(I)c2)c(I)c1)C(=O)ON1C(=O)CCC1=O ; NCCc1cc(I)c(Oc2cc(I)c(O)c(I)c2)c(I)c1]

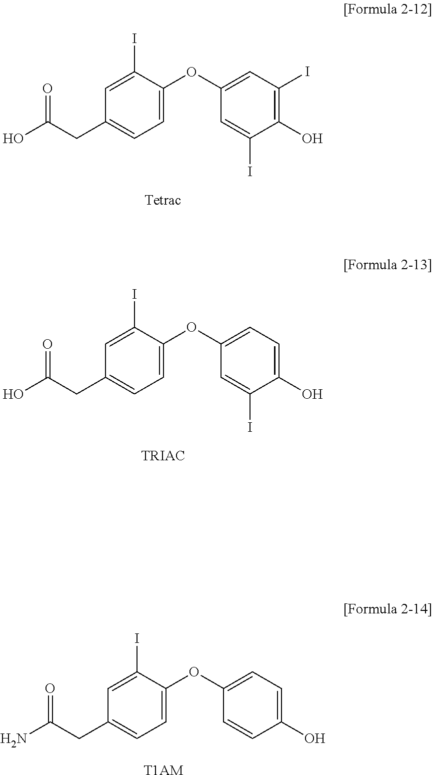

The TTR ligand may be conjugated to an adeno-associated virus through a specific residue of a specific amino acid present on the capsid of the adeno-associated virus.

### SPECIFIC EXAMPLES

Hereinafter, specific examples of the vector for delivering an active substance of the present application will be described.

As a specific example of the present application,
in Formula 1,
when R′ is

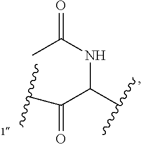 [SMILES: *C(=O)C(*)NC(C)=O]

a vector for delivering an active substance in which one or more TTR ligands are conjugated to the surface of an adeno-associated virus, represented by the following Formula 1-2, is provided:

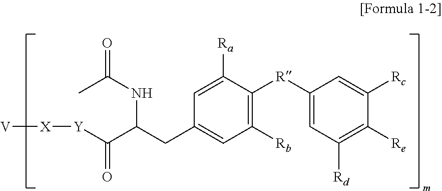

wherein V is an adeno-associated virus, V—X— is named an adeno-associated virus moiety, and the adeno-associated virus moiety is derived from an adeno-associated virus.

In this case, X is —NH— or —S—, and wherein X is derived from a residue of an amino acid located on the capsid of an adeno-associated virus,
Y is -(L)n-, —C(═O)-(L)n-, —C(═NH₂⁺)-(L)n-, —S-(L)n-, or

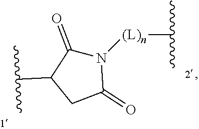

wherein 1′ is linked to X and 2′ is linked to R′,
wherein, L is a linker moiety, and L is substituted or unsubstituted C₁₋₂₀ alkylene, substituted or unsubstituted C₂₋₂₀ alkenylene, substituted or unsubstituted C₂₋₂₀ alkynylene, or substituted or unsubstituted C₁₋₂₀ heteroalkylene, substituted or unsubstituted C₂₋₂₀ heteroalkenylene, or substituted or unsubstituted C₂₋₂₀ heteroalkynylene,
wherein, the heteroalkylene, heteroalkenylene, or heteroalkynylene includes one or more heteroatom groups selected from the group consisting of —NH—, —O—, —S—, —O—N═, —S(═O)—, and —S(═O)₂—, or one or more heteroatoms selected from the group consisting of O, N, and S,
wherein, the substitution refers to being substituted with one or more non-hydrogen substituents, wherein the non-hydrogen substituent is any one selected from the group consisting of —R¹, —C(R¹)₃, —OR¹, —SR¹, —N(R¹)₂, —N═C═O, —NCS, —NO, —NO₂, ═N₂, —N₃, —NHC(═O)R¹, ═O, ═S, —C(═O)R¹, —C(═O)OR¹, —C(═O)NR¹R¹, —S(═O)₂R¹, —OS(═O)₂OR¹, —S(═O)₂NR¹, —S(═O)R¹, alkylene-C(═O)R¹, —C(═S)R¹, alkylene-C(═O)OR¹, —C(═S)OR¹, —C(═O)SR¹, —C(═S)SR¹, —C(═O)NR¹R¹, alkylene-C(═O)NR¹R¹, —C(═S)NR¹R¹, and —C(-NR¹)NR¹R¹,
wherein, R¹ is H, halogen, C₁₋₆ alkyl, C₂₋₆ alkene, C₂₋₆ alkyne, C₁₋₆ heteroalkyl, C₆₋₁₂ aryl, C₇₋₁₂ arylalkyl or C₃₋₁₀ cycloalkyl, C₃₋₁₀ cycloalkene, C₃₋₁₀ cycloalkyne, or C₃₋₁₀ heterocycloalkyl,
wherein, n is 0 or 1,
Ra is H, C₁₋₃ alkyl, or I,
Rb is H, C₁₋₃ alkyl, or I,
R″ is —O—, —S—, —NH—, or —CH₂—,
Rc is H, C₁₋₃ alkyl, or I,
Rd is H, C₁₋₃ alkyl, or I, and
Re is OH, and
m is an integer of 2 or more.

In an embodiment, in this case, L may be substituted or unsubstituted C₁₋₁₀ alkylene, substituted or unsubstituted C₂₋₁₀ alkenylene, substituted or unsubstituted C₂₋₁₀ alkynylene, substituted or unsubstituted C₁₋₁₀ heteroalkylene, substituted or unsubstituted C₂₋₁₀ heteroalkenylene, or substituted or unsubstituted C₂₋₁₀ heteroalkynylene.

In an embodiment, in this case, L may be substituted or unsubstituted C₁₋₅ alkylene, substituted or unsubstituted C₂₋₅ alkenylene, substituted or unsubstituted C₂₋₅ alkynylene, substituted or unsubstituted C₁₋₅ heteroalkylene, substituted or unsubstituted C₂₋₅ heteroalkenylene, or substituted or unsubstituted C₂₋₅ heteroalkynylene.

In an embodiment, in this case, the residue of an amino acid located on the capsid of the adeno-associated virus may be an amine residue of lysine or a thiol residue of cysteine.

In an embodiment, in this case, the residue of an amino acid located on the capsid of the adeno-associated virus may be an amine residue of lysine.

As a specific example of the present application, in Formula 1,
when X is —NH—,
R′ is

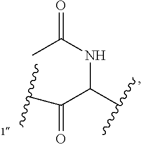 [SMILES: *C(=O)C(*)NC(C)=O]

Y is -(L)n-, n is 0, and
R″ is O,
a vector for delivering an active substance in which one or more TTR ligands are conjugated to the surface of an adeno-associated virus, represented by the following Formula 1-3, is provided:

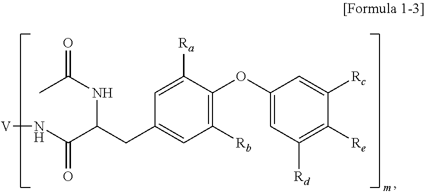

wherein V—NH— is an adeno-associated virus moiety, and wherein the adeno-associated virus moiety is derived from an adeno-associated virus,
in the adeno-associated virus moiety, —NH— is derived from the amine residue of lysine located on the capsid of an adeno-associated virus,
Ra is H, methyl, or I,
Rb is H, methyl, or I,
Rc is H, C₁₋₃ alkyl, or I,
Rd is H, C₁₋₃ alkyl, or I,
Re is OH, and
m is an integer of 2 or more.

As a specific example of the present application, in Formula 1,
when X is —NH—,
R′ is

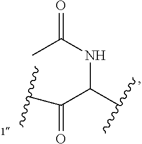 [SMILES: *C(=O)C(*)NC(C)=O]

Y is -(L)n-, and n is 0,
Ra, Rb, Rc, and Rd are each I,
Re is OH, and
R″ is O,
a vector for delivering an active substance in which one or more TTR ligands are conjugated to the surface of an adeno-associated virus, represented by the following Formula 1-4, is provided:

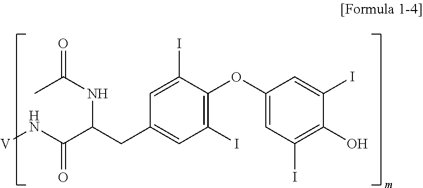

wherein V—NH— is an adeno-associated virus moiety, and wherein the adeno-associated virus moiety is derived from an adeno-associated virus,
wherein in the adeno-associated virus moiety, —NH— is derived from the amine residue of lysine located on the capsid of the adeno-associated virus, and
wherein m is an integer of 2 or greater.

As a specific example of the present application, a vector for delivering an active substance in which one or more TTR ligands are conjugated to the surface of an adeno-associated virus, represented by the following Formula 1-5, is provided:

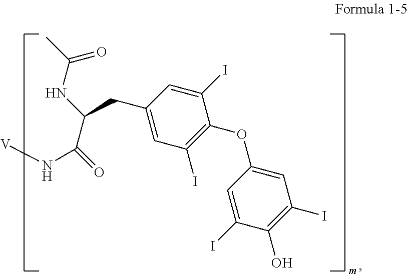

wherein V—NH— is an adeno-associated virus moiety, and wherein the adeno-associated virus moiety is derived from an adeno-associated virus,
wherein in the adeno-associated virus moiety, —NH— is derived from the amine residue of lysine present on the capsid of an adeno-associated virus, and
wherein m is an integer of 2 or greater.

The present application provides a method for preparing a vector for delivering an active substance in which one or more TTR ligands are conjugated to the surface of an adeno-associated virus.

In an embodiment, the vector for delivering an active substance of the present application may be prepared by a method including contacting a TTR ligand and an adeno-associated virus.

In this case, the vector may be produced by reacting the conjugating part of the TTR ligand with a residue of an amino acid present on the capsid of the adeno-associated virus. In an example, the vector may be produced by reacting an amine residue of lysine or a thiol residue of cysteine present on the capsid of an adeno-associated virus with the conjugating part of the TTR ligand.

In an embodiment, the vector for delivering an active substance of the present application may be produced by a method including mixing a first composition including a TTR ligand represented by Formula 2-9 or 2-10 with a second composition including an adeno-associated virus.

In this case, the ratio of the number of moles of TTR ligand included in the first composition and the viral genome titer of the adeno-associated virus included in the second composition may be 50 to 100,000:1 to 10. For example, the ratio may be about 50:1, 100:1, 1000:1, 10000:1, or 100000:1.

According to an embodiment, a specific example of the vector for delivering an active substance provided by the present application may be produced by the reaction of the following reaction scheme.

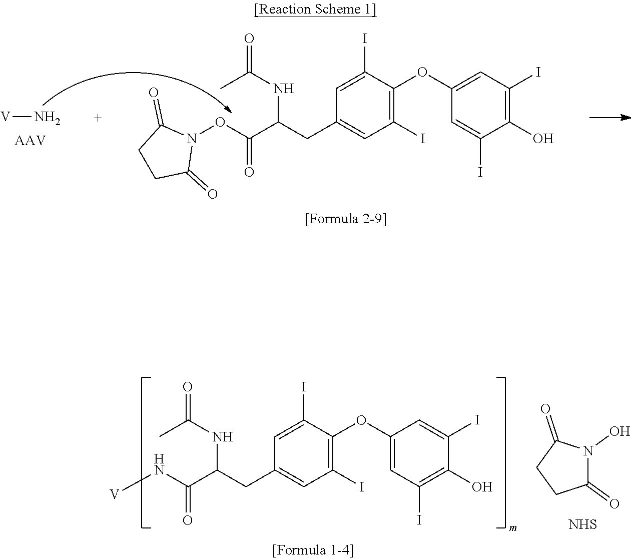

According to an embodiment of the present application, as shown in Reaction Scheme 1, the vector for delivering an active substance of the present application is produced by reacting the amine residue of lysine located on the capsid of an adeno-associated virus with the conjugating part of a TTR ligand. More specifically, in this reaction, the amine group which is a nucleophile located on the capsid of the adeno-associated virus attacks the carbon of the ester group of the TTR ligand, and in this case, the vector for delivering an active substance of the present application is produced, and N-hydroxysuccinimide is released.

According to an embodiment, a specific example of the vector for delivering an active substance provided by the present application may be prepared by the reaction of the following reaction scheme.

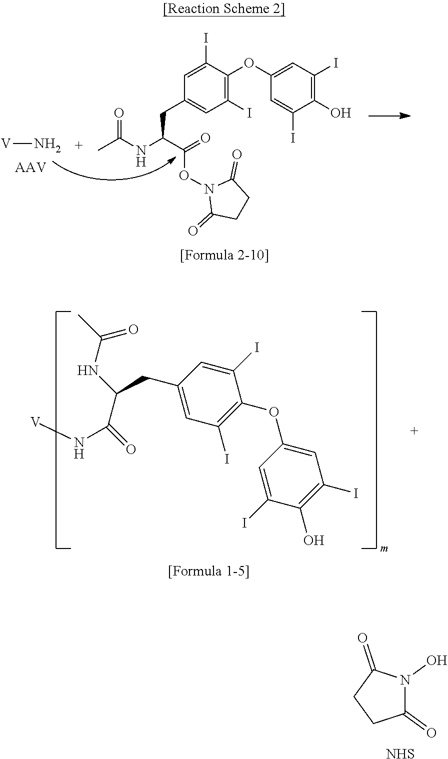

According to an embodiment, a specific example of a vector for delivering an active substance provided by the present application may be prepared by reacting the TTR ligand represented by Formula 2-9 or Formula 2-10 with an adeno-associated virus.

In this case, the TTR ligand represented by Formula 2-9 or Formula 2-10 may be prepared by a method including:
(i) preparing N-acetyl thyroxin by contacting T4 (thyroxine) and N-acetyl hydroxysuccinimide; and
(ii) contacting N-acetyl thyroxin and N-hydroxy.

In the (i), N-acetyl thyroxin is prepared by reacting T4 with N-acetyl hydroxysuccinimide. In this case, the amine group present at one end of T4 is capped with a ketone group.

In this case, when the amine group at one end of T4 is not capped with a ketone group, there is a possibility that the amine group at one end of T4, which does not react during the conjugating of the TTR ligand to the adeno-associated virus vector, and the carboxyl group of an obtained product will react again.

Further, when the amine group at one end of T4 is not capped with a ketone group and the carboxyl group of T4 is directly substituted with an N-hydroxysuccinimide ester (NHS ester) group, a problem in that the T4 is not sufficiently dissolved in a solvent may occur. In addition, in this case, the TTR ligand produced from T4 may have the property of a zwitterion, which may have a negative impact on the subsequent reaction between the TTR ligand and the adeno-associated virus.

Therefore, as in an embodiment of the present application, it may be a preferable method for producing the vector for delivering an active substance of the present application to cap the amine group at one end of T4 with a ketone group and substitute a carboxyl group at one end of T4 with an NHS ester group.

An example of a method for producing the TTR ligand represented by Formula 2-10 is described through Examples.

The vector for delivering an active substance of the present application may include at least one or more active substances.

Hereinafter, an active substance which may be included in the vector for delivering an active substance of the present application will be described.

### Active Substance

In the present application, the term “active substance” refers to a material which is introduced into a vector and delivered in order to obtain a desired effect.

In an example, the active substance may be delivered to a target site by being included in a viral particle of the adeno-associated virus moiety.

In an embodiment, the active substance may be one or more materials selected from a nucleic acid (DNA, RNA, PNA, and the like), a fluorescent material, a radioactive agent, a drug, and a drug precursor. The nucleic acid may be a nucleic acid or gene encoding a specific protein or peptide.

In an embodiment, two or more active substances, which are the same or different, may be introduced into one or more vectors independently or together.

In one embodiment, the nucleic acid may be a nucleic acid associated with a CRISPR/Cas system and/or nucleic acid encoding a protein associated with a CRISPR/Cas system, but is not limited thereto. As an example, the nucleic acid associated with the CRISPR/Cas system may be one or more RNAs selected from crRNA, tracrRNA, and guide RNA, or DNA encoding the same, but is not limited thereto. As an example, the protein associated with the CRISPR/Cas system may be a Cas protein. The Cas protein may be any one selected from Cas9, Cas12, and Cas13, but is not limited thereto.

In an embodiment, the active substance may be a transgene.

In an embodiment, the transgene may be any one selected from a DNA, a DNA fragment, cDNA, a cDNA fragment, RNA, and an RNA fragment.

In an embodiment, the transgene may be a DNA, a DNA fragment, cDNA, or a fragment of a cDNA, which encodes a protein or a fragment of a protein.

In an embodiment, the DNA, DNA fragment, cDNA or fragment of cDNA may include all or part of a sequence encoding a functional and/or structural part of an RNA molecule. This RNA molecule is selected from the group consisting of, for example, ribosomal RNA (rRNA), transfer RNA (tRNA), small nuclear RNA (snRNA), small nucleolar RNA, microRNA (miRNA), long noncoding RNA, short interfering RNA, guide RNA, crRNA (CRISPR RNA), and any functional RNA.

In an embodiment, the RNA or RNA fragment may include part or all of the sequences that constitute the RNA molecule. This RNA molecule is selected from the group consisting of, for example, ribosomal RNA (rRNA), transfer RNA (tRNA), small nuclear RNA (snRNA), small nucleolar RNA, microRNA (miRNA), long noncoding RNA, short interfering RNA, guide RNA, crRNA (CRISPR RNA), and any functional RNA.

In a specific example, the transgene may be any one selected from the group consisting of MECP2, SCN1A, NF2, SNCA, LRRK2, APP, Tau, Nav1.7, C₉rof72, SOD1, DYRK1A, IT15, HTT, HEXA, RA11, PRGN, UBE3A, ABCA4, RP1, PAX6, USH2A, NRP1, SERPINA1, PCSK9, LIPA, HFE, ALAS1, ATP7B, COL4A5, LDHA, HAO1, DUX4, DMPK, DMD, BCL11A, Mex3B, FVIII, CIDEC, SCD1, GNB3, SMN1, SMN2, GAA, FGFR3, CLCN7, PMP22, CFTR, ENAC, GHR, TTR, APOEε4, APOEε3, APOEε2, MAPT, GRN, AADC, GBA1, ASPA, CLN2, GLB1, SGSH, NAGLU, IDS, NPC1, SCA1(ATXN1), SCA3(ATXN3), SCA7(ATXN7), TARDBP(TDP-43), FRDA(FXN), SCN9A, SCN10A, GAN, 3R tau, 4R tau, AARS, ABCD1, ACOX1, ADGRV1, ADRA2B, AGA, AGER, ALDH7A1, ALG13, ALS2, ANG, ANXA11, ARHGEF9, ARSA, ARSB, ARV1, ASAH1, ATN1, ATP10A, ATP13A2, ATXN1, ATXN2, ATXN3, BAX, BCL-2, BDNF, BICD2, CACNA1A, CACNA1H, CACNB4, CASR, CCNF, CDKL5, CERS1, CFAP410, CHCHD10, CHD2, CHMP2B, CHRNA2, CHRNA4, CHRNA7, CHRNB2, CLCN2a, CLN1, CLN3, CLN5, CLN6, CLN8, CNTN2, CPA6, CSTB, CTNS, CTSA, CTSD, DAO, DCTN1, DEPDC5, DNAJB2, DNM1, DOCK7, DRD2, DYNC1H1, EEF1A2, EFHC1, EGLN1, EPHA4, EPM2A, ERBB4, FGF12, FRRS1L, FTL, FUCA1, FUS, FAXN, GABRA, GABRB1, GABRB3, GABRD, GABRG2, GAL, GALC, GALNS, GBA, GFAP, GLA, GLE1, GLT8D1, GNAO1, GNS, GOSR2, GPR98, GRIA1, GRIA2, GRIK1, GRIN1, GRIN2A, GRIN2B, GRIN2D, GSTM1, GUF1, GUSB, HCN1, HGSNAT, HNRNPA1, HYAL1, IDUA, IGHMBP2, IL-1, ITPA, JPH3, KCNA2, KCNB1, KCNC1, KCNMA1, KCNQ2, KCNQ3, KCNT1, KCTD7, LAL, LAMP2, LG11, LMNB2, MAN2B1, MAN2B2, MAN2C1, MANBA, MATR3, MBD5, MFSD8, NAGA, NECAP1, NEFH, NEK1, NEU1, NHLRC1, NPC2, NR4A2, NTRK2, OCA2, OPTN, PARK2, PARK7, PCDH19, PEX1, PEX2, PEX3, PEX5, PEX6, PEX10, PEX11B, PEX12, PEX13, PEX14, PEX16, PEX19, PEX26, PFN1, PINK1, PLCB1, PNPO, PON1, PON2, PON3, PPARGC1A, PRDM8, PRICKLE1, PRKN, PRNP, PRPH, PRRT2, PSAP, S106P, SCARB2, SCN1B, SCN2A, SCN8A, SCN9Ab, SETX, SGSH, SIGMAR1, SK, SKP1, SLC1A1, SLC1A2, SLC2A1, SLC6A, SLC9A6, SLC12A5, SLC13A5, SLC25A12, SLC25A22, SLCA17A5, SMPD1, SNRPN, SPG11, SPTAN1, SQSTM1, ST3GAL3, ST3GAL5, STX1B, STXBP1, SYP, SYT1, SZT2, TAF5, TBC1D24, TBCE, TBK1, TBP, TITF-1, TREM2, UBA5, UBE1, UBQLN2, UCH-L1, UNC13A, VAPB, VCP, VPS35, WWOX, and XBP1, but is not limited thereto.

In an embodiment, the transgene may be a gene associated with the treatment of a neurological disorder or a gene encoding a protein associated with the treatment of a neurological disorder, but is not limited thereto. In this case, examples of the gene associated with the treatment of the neurological disorder may be MECP2, SCN1A, NF2, SNCA, LRRK2, APP, Tau, Nav1.7, C9rof72, SOD1, DYRK1A, IT15, HTT, HEXA, RA11, PRGN, UBE3A, ABCA4, RP1, PAX6, USH2A, NRP1, SMN1, SMN2, GAA, PMP22, TTR, APOEε4, APOEε3, APOEε2, MAPT, GRN, AADC, GBA1, ASPA, CLN2, GLB1, SGSH, NAGLU, IDS, NPC1, SCA1(ATXN1), SCA3(ATXN3), SCA7(ATXN7), TARDBP(TDP-43), FRDA(FXN), SCN9A, SCN10A, or GAN.

### Composition

The present application provides a composition including the above-described vector for delivering an active substance.

The composition is used to deliver the active substance included in the vector to a desired target site.

In one embodiment, the present application provides a composition for delivering an active substance to a target site. In this case, the composition may include a vector for delivering an active substance of the present application and the active substance. In this case, the vector for delivering an active substance of the present application may be any one selected from the vectors for delivering an active substance represented by Formula 1 and Formulae 1-2 to 1-5.

In one embodiment, the target site may be the central nervous system, the peripheral nervous system, or nerve cells, but is not limited thereto. Preferably, the target site may be the central nervous system.

In one embodiment, the target site may be the brain, spinal cord, or nerve cells, but is not limited thereto. The brain includes the cerebrum, cerebellum, and brainstem, but is not limited thereto. The cerebrum includes the frontal lobe, parietal lobe, temporal lobe, occipital lobe, and hippocampus. The brainstem includes the diencephalon, midbrain, pons, and medulla. The diencephalon includes the thalamus, hypothalamus, pituitary gland, and pineal gland. Further, the brain includes the striatum, substantia nigra, parietal cortices, and globus pallidus. The brain includes the forebrain, midbrain, and hindbrain, but is not limited thereto. The forebrain includes the frontal lobe, temporal lobe, parietal lobe, occipital lobe, and olfactory bulb, but is not limited thereto. The midbrain includes the colliculi, tegmentum, and cerebral peduncles, but is not limited thereto. The hindbrain includes the medulla, pons, and cerebellum, but is not limited thereto. In addition, the brain includes nerve cells of the brain.

In one embodiment, the target site may be the deep brain.

That is, the composition of the present application may be delivered to any one of the specific tissues of the brain described above, but is not limited thereto. The target site may be any one of the specific tissues of the brain described above, but is not limited thereto.

In one embodiment, the target site may be the peripheral nervous system. The peripheral nervous system may be the sciatic nerve, femoral nerve, tibial nerve, peroneal nerve, phrenic nerve, radial nerve, saphenous nerve, sural nerve, or ulnar nerve, but is not limited thereto.

### Pharmaceutical Composition and Treatment Method Using the Same

In a specific example, the present application provides a pharmaceutical composition including the vector for delivering an active substance of the present application. In this case, the active substance is an agent that exhibits a therapeutic effect. In this case, the vector for delivering an active substance of the present application may be any one selected from the vectors for delivering an active substance represented by Formula 1 and Formulae 1-2 to 1-5.

In an embodiment, the pharmaceutical composition of the present application may be a pharmaceutical composition for treating a target disease. In this case, the target disease may be a neurological disorder.

In this case, the target disease may be a neurological disorder. In this case, the neurological disorder may be a lysosomal storage disorder.

In an embodiment, the neurological disorder may be a central nervous system disorder. In an embodiment, the pharmaceutical composition of the present application may be a pharmaceutical composition for treating a central nervous system disorder.

In an embodiment, the neurological disorder may be a peripheral nervous system disorder. In an embodiment, the pharmaceutical composition of the present application may be a pharmaceutical composition for treating a peripheral nervous system disorder.

In an embodiment, the central nervous system disorder may be any one selected among neurological disorders that affect the structure or function of the brain or spinal cord. In an embodiment, the central nervous system disorder may be any one or more selected from a vascular disorder, a disorder caused by infection, a structural disorder, a functional disorder, a neurodegenerative disorder, a mood disorder, a neurodevelopmental disorder, and a psychiatric disorder, but is not limited thereto.

In an embodiment, the vascular disorder includes stroke, transient ischemic attack (TIA), subarachnoid hemorrhage, subdural hemorrhage, hematomas, extradural hemorrhage, and the like, but is not limited thereto.

In an embodiment, the disorder caused by infection includes meningitis, encephalitis, polio, and epidural abscesses, but is not limited thereto. In an embodiment, the structural disorder includes brain or spinal cord injuries, Bell's palsy, cervical spondylosis, carpal tunnel syndrome, brain or spinal cord tumors, peripheral neuropathy, and Guillain-Barre syndrome, but is not limited thereto. In an embodiment, the functional disorder includes headache, epilepsy, dizziness, and neuralgia, but is not limited thereto.

In an embodiment, the neurodegenerative disorder includes Alzheimer's disease, giant axonal neuropathy, GM2 gangliosidosis, CLN1 disease, GM2 AB variant, SURF1-associated Leigh syndrome, adult polyglucosan body disease (APBD), Lafora disease, tauopathies, glycogen storage disease (GSDs), Huntington's disease, Parkinson's disease, multiple sclerosis, and amyotrophic lateral sclerosis (ALS), but is not limited thereto. In an embodiment, the mood disorder includes a bipolar disorder, a depressive disorder, and an anxiety disorder, but is not limited thereto.

In an embodiment, the neurodevelopmental disorder includes attention deficit hyperactivity disorder (ADHD), developmental language disorder (DLD), autism spectrum disorder (ASD), intellectual disabilities (IDs), intellectual development disorder (IDD), tic disorder, motor abnormalities, apraxia, Angelman syndrome, Prader-Willi syndrome, FOXG1 syndrome, inborn error of metabolism, inherited metabolism disorders, Fragile X syndrome, Down syndrome, Rett syndrome, neurogenetic disorders such as hypogonadotropic/hypogonadal syndromes, dyslexia, dyscalculia, disorders caused by neurotoxic materials, disorders caused by heavy metals, and the like, but is not limited thereto.

In an embodiment, the psychiatric disorder includes schizophrenia, but is not limited thereto.

In an embodiment, the central nervous system disorder may be any one among central nervous system disorders disclosed in US Publication Patent No. US 2021/0162072 A1.

In an embodiment, the central nervous system disorder may be any one or more selected from hereditary epilepsy, SLC6A1 haploinsufficiency disorder, SLC13A5 deficiency, Dravet syndrome, neurofibromatosis type 2, epileptic encephalopathy, chronic pain, Tay-Sachs disease, Protocki-Lupski syndrome, frontotemporal dementia, Stargardt disease, retinitis pigmentosa, aniridia, Usher syndrome, wet AMD, dry AMD, Pompe disease, spinal muscular atrophy (SMA), type I SMA, type II SMA, transthyretin amyloidosis, AADC deficiency, Canavan disease, late infantile neuronal ceroid lipofuscinosis, gangliosidosis 1, mucopolysaccharidosis (MPS), MPS II, MPS 1, mucopolysaccharidosis IIIA and IIIB, mucopolysaccharidosis IIA, Niemann-Pick C1, spinocerebellar ataxia 1, spinocerebellar ataxia 3, spinocerebellar ataxia 7, type I, 11 and III spinal muscular atrophy, Friedreich ataxia, post-herpetic or trigeminal neuralgia, Batten disease, metachromatic leukodystrophy, and developmental and epileptic encephalopathy (KCNQ2).

In an embodiment, the lysosomal storage disorder may be any one or more selected from metachromatic leukodystrophy (MLD), MPS 1, MPS 1-H, MPS II, MPS IIIA, MPS IIIB, MPSVII, cystinosis, Fabry disease, Gaucher disease, GM1 gangliosidosis, GM2 gangliosidosis, Krabbe disease, MSD, Pompe disease, CLN1 Batten disease, CLN2 Batten disease, CLN3 Batten disease, and Niemann-Pick disease, but is not limited thereto.

In an embodiment, the peripheral nervous system disorder may be any one selected from giant axonal neuropathy and Charcot-Marie-Tooth disease, but is not limited thereto.

In one embodiment, the pharmaceutical composition provided by the present application may further include a pharmaceutically acceptable additive (excipient). The additive may be any appropriately selected from substances well known in the art depending on the purpose of use.

Furthermore, the pharmaceutical composition may further include another therapeutically active substance (for example, a therapeutic agent) for combination treatment. For example, the pharmaceutical composition may further contain one or more other therapeutic agents suitable for treating cancer (peripheral nerve sheath tumor schwannoma, head and neck cancer, glioblastoma, and neurofibromatosis), or a nervous system disorder.

In one embodiment, the pharmaceutical composition provided by the present application may be administered in various dosage forms by oral and/or parenteral methods during clinical administration. Preferably, the pharmaceutical composition may be administered in an injection form for the treatment of a neurological disorder.

In one embodiment, the pharmaceutical composition of the present application may be used in a parenteral administration method. In one embodiment, a formulation for parenteral administration including the pharmaceutical composition of the present application may be prepared and/or used.

The parenteral administration method may be any injection method selected from intrathecal (IT) injection, intracerebroventricular (ICV) injection, intracranial injection, subcutaneous injection, intravenous injection, intramuscular injection, injection within the substantia nigra pars compacta and the ventral tegmental area, intraparenchymal injection, cisterna magna injection, and intrathoracic injection, or combinations thereof. Preferably, an intrathecal injection or intracerebroventricular injection method may be used.

The formulation for parenteral administration may be prepared as a solution or suspension by being mixed with a stabilizer or buffer in water, and the preparation may be prepared in a unit dosage form in ampoules or vials. Further, the formulation for parenteral administration may include an adjuvant such as a preservative, a stabilizer, a hydrating agent, or an emulsion-promoting agent, a salt and/or buffer for adjusting osmotic pressure, and other therapeutically useful materials, and the formulation may be formulated by mixing, granulation, or coating as a typical method. In addition, the pharmaceutical composition according to the present application may be prepared with a formulation such as polymeric excipient and the like, for the sustained release of a vector for delivering an active substance and/or active substance.

In another embodiment, the pharmaceutical composition of the present application may be used in an oral administration method. In one embodiment, a formulation for oral administration including the pharmaceutical composition of the present application may be prepared and/or used.

In one embodiment, a preferred dose of the pharmaceutical composition of the present application may be appropriately selected depending on the condition and body weight of a patient, the severity of a symptom, the form of the drug, the administration route, and the duration. For example, the pharmaceutical composition of the present application may be administered to a patient in an appropriate amount, such that 1×10¹¹ vg/kg to 1×10¹⁵ vg/kg of the vector for delivering an active substance of the present application is administered. Preferably, the pharmaceutical composition of the present application may be administered to a patient in an appropriate amount, such that 1×10¹² vg/kg to 1×10¹³ vg/kg of the vector for delivering an active substance of the present application is administered. More specifically, the pharmaceutical composition of the present application may be administered to a patient in an appropriate amount, such that about 1×10¹¹ vg/kg, 2×10¹¹ vg/kg, 3×10¹¹ vg/kg, 5×10¹¹ vg/kg, 7×10¹¹ vg/kg, 1×10¹² vg/kg, 5×10¹² vg/kg, or 1×10¹³ vg/kg of the vector for delivering an active substance of the present application is administered.

As another example, the active ingredient of the pharmaceutical composition of the present application may be administered at 0.2 mg/kg to 200 mg/kg per day. Specifically, the active ingredient of the pharmaceutical composition of the present application may be administered at about 0.2 mg/kg, 0.4 mg/kg, 0.6 mg/kg, 0.8 mg/kg, 1 mg/kg, 3 mg/kg, 5 mg/kg, 10 mg/kg, 20 mg/kg, 40 mg/kg, 80 mg/kg, 120 mg/kg, 160 mg/kg, or 200 mg/kg per day.

Furthermore, the pharmaceutical composition of the present application may be administered once a day or divided into several doses, but is not limited thereto.

In another specific example, the present application provides a method for treating a neurological disorder, the method including administering a pharmaceutical composition including a vector for delivering an active substance of the present application to a subject. In this case, the neurological disorder may be a disorder of the central nervous system or the peripheral nervous system.

In one embodiment, a method for treating a neurological disorder in a subject is provided, the method including administering the pharmaceutical composition of the present application into the cerebrospinal fluid of the subject.

In this case, the administration into the cerebrospinal fluid of the subject may be performed through intrathecal injection or intracerebroventricular injection, but is not limited thereto.

The neurological disorder of the subject may be a neurological disorder of a mammal, and in this case, the mammal includes a human, a canine, a feline, a rodent, a cow, a horse, and a pig, but is not limited thereto. As an example, the mammal may be a human. As another example, the mammal may be an animal other than a human.

### Diagnostic Composition and Diagnostic Method Using the Same

Further, the present application provides a method for diagnosing a neurological disorder in a subject through a diagnostic composition including a vector for delivering an active substance. In this case, the neurological disorder may be a central nervous system disorder or a peripheral nervous system disorder. In this case, the active substance may be a labeling substance, for example, a fluorescent agent or a radioactive substance, which may be used to diagnose a neurological disorder.

A specific embodiment of the present application will be described by way of example, below.

As an example, a method for delivering an active substance to the nervous system of a subject is provided, wherein the method includes administering the vector for delivering an active substance of the present application or a composition including the same into the cerebrospinal fluid of the subject. In this case, the nervous system may be the central nervous system or the peripheral nervous system.

In this case, the administration into the cerebrospinal fluid of the subject may be performed through intrathecal injection or intracerebroventricular injection, but is not limited thereto.

As another example, a method for diagnosing a neurological disorder in a subject is provided, wherein the method includes administering the composition or pharmaceutical composition of the present application into the cerebrospinal fluid of the subject. In this case, the neurological disorder may be a central nervous system disorder or a peripheral nervous system disorder.

In this case, the administration into the cerebrospinal fluid of the subject may be performed through intrathecal injection or intracerebroventricular injection, but is not limited thereto.

### SPECIFIC EMBODIMENTS OF PRESENT APPLICATION

Hereinafter, various embodiments provided by the present application will be described. However, although the embodiments described below correspond to an example that may be provided by the present application, the present application is not limited to the following embodiments.

### Embodiment 1

A vector for delivering an active substance in which one or more TTR ligands are conjugated to the surface of an adeno-associated virus, represented by the following Formula 1:

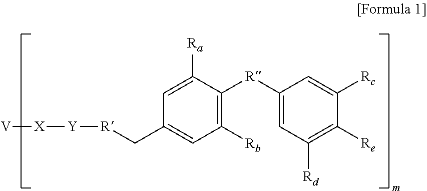

wherein V is an adeno-associated virus, V—X— is named an adeno-associated virus moiety, and the adeno-associated virus moiety is derived from an adeno-associated virus.

In this case, X is —NH— or —S—, and wherein, X is derived from a residue of an amino acid located on the capsid of an adeno-associated virus,
Y is -(L)n-, —C(═O)-(L)n-, —C(═NH₂⁺)-(L)n-, —S-(L)n-, or

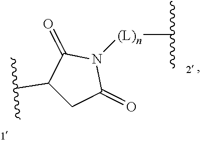

wherein 1′ is linked to X and 2′ is linked to R′,
wherein L is a linker moiety, and L is a substituted or unsubstituted C₁₋₂₀ alkylene, substituted or unsubstituted C₂₋₂₀ alkenylene, substituted or unsubstituted C₂₋₂₀ alkynylene, substituted or unsubstituted C₁₋₂₀ heteroalkylene, substituted or unsubstituted C₂₋₂₀ heteroalkenylene, or substituted or unsubstituted C₂₋₂₀ heteroalkynylene,
wherein the heteroalkylene, heteroalkenylene, or heteroalkynylene includes one or more heteroatom groups selected from the group consisting of —NH—, —O—, —S—, —O—N═, —S(═O)—, and —S(═O)₂—, or one or more heteroatoms selected from the group consisting of O, N, and S,
wherein the substitution refers to being substituted with one or more non-hydrogen substituents, and the non-hydrogen substituent is any one selected from the group consisting of —R¹, —C(R¹)₃, —OR¹, —SR¹, —N(R¹)₂, —N═C═O, —NCS, —NO, —NO₂, ═N₂, —N₃, —NHC(═O)R¹, ═O, ═S, —C(═O)R¹, —C(═O)OR¹, —C(═O)NR¹R¹, —S(═O)₂R¹, —OS(═O)₂OR¹, —S(═O)₂NR¹, —S(═O)R¹, alkylene-C(═O)R¹, —C(═S)R¹, alkylene-C(═O)OR¹, —C(═S)OR¹, —C(═O)SR¹, —C(═S)SR¹, —C(═O)NR¹R¹, alkylene-C(═O)NR¹R¹, —C(═S)NR¹R¹, and —C(—NR¹)NR¹R¹,
wherein R¹ is H, halogen, C₁₋₆ alkyl, C₂₋₆ alkene, C₂₋₆ alkyne, C₁₋₆ heteroalkyl, C₆₋₁₂ aryl, C₇₋₁₂ arylalkyl or C₃₋₁₀ cycloalkyl, C₃₋₁₀ cycloalkene, C₃₋₁₀ cycloalkyne, or C₃₋₁₀ heterocycloalkyl,
wherein n is 0 or 1,
R′ is —C(═O)—, C₁₋₃ alkylene,

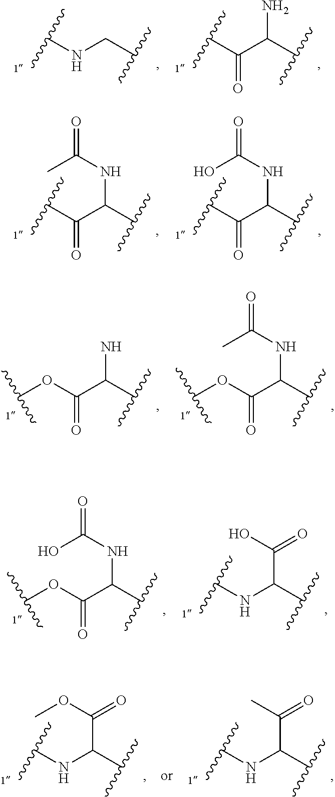

wherein 1″ is linked to Y,
Ra is H, C₁₋₃ alkyl, or I,
Rb is H, C₁₋₃ alkyl, or I,
R″ is —O—, —S—, —NH—, or —CH₂—,
Rc is H, C₁₋₃ alkyl, or I,
Rd is H, C₁₋₃ alkyl, or I,
Re is OH, and
m is an integer of 2 or greater.

Embodiment 2. The vector for delivering an active substance of Embodiment 1, wherein when n is 1, L is (—OCH₂CH₂—)q, wherein q is an integer between 1 and 10.

Embodiment 3. The vector for delivering an active substance of Embodiment 1, wherein the residue of an amino acid located on the capsid of the adeno-associated virus is an amine residue of lysine or a thiol residue of cysteine.

Embodiment 4. The vector for delivering an active substance of Embodiment 1, wherein the residue of an amino acid located on the capsid of the adeno-associated virus is an amine residue of lysine.

### Embodiment 5

The vector for delivering an active substance of Embodiment 1, wherein the vector for delivering an active substance in which one or more TTR ligands are conjugated to the surface of the adeno-associated virus is represented by formula 1-2:

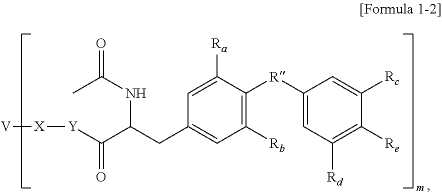

wherein V, X, Y, R″, Ra, Rb, Rc, Rd, Re, and m are the same as defined in Embodiment 1.

Embodiment 6. The vector for delivering an active substance of Embodiment 5, wherein X is —NH—, wherein X is derived from an amine residue of lysine located on the capsid of the adeno-associated virus.

Embodiment 7. The vector for delivering an active substance of Embodiment 6, wherein when n is 1, L includes q ethylene glycol units, wherein q is an integer between 1 and 10.

Embodiment 8. The vector for delivering an active substance of Embodiment 6, wherein when n is 1, L is (—OCH₂CH₂—)q, wherein q is an integer between 1 and 10.

Embodiment 9. The vector for delivering an active substance of Embodiment 7 or 8, wherein q is an integer between 1 and 5.

Embodiment 10. The vector for delivering an active substance of Embodiment 1, wherein the vector for delivering an active substance in which one or more TTR ligands are conjugated to the surface of adeno-associated virus is represented by the following Formula 1-3:

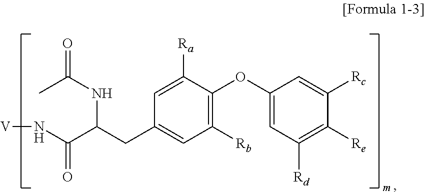

wherein V, Ra, Rb, Rc, Rd, Re, and m are the same as defined in Embodiment 1.

Embodiment 11. The vector for delivering an active substance of Embodiment 1, wherein the vector for delivering an active substance in which one or more TTR ligands are conjugated to the surface of the adeno-associated virus is represented by the following Formula 1-4:

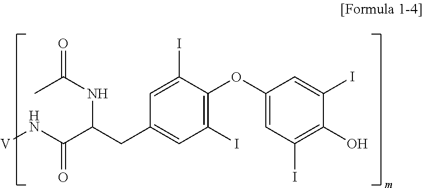

wherein V and m are the same as defined in Embodiment 1.

Embodiment 12. The vector for delivering an active substance of Embodiment 1, wherein the vector for delivering an active substance in which one or more TTR ligands are conjugated to the surface of the adeno-associated virus is represented by the following Formula 1-5:

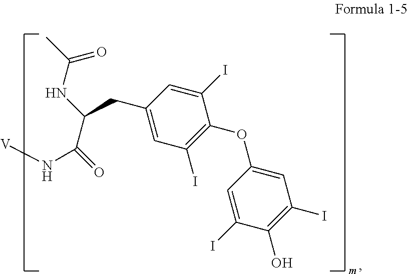

wherein V and m are the same as defined in Embodiment 1.

Embodiment 13. The vector for delivering an active substance of any one of Embodiments 10 to 12, wherein in V-NH—, the —NH— is derived from an amine residue of lysine located on the capsid of the adeno-associated virus.

Embodiment 14. The vector for delivering an active substance of any one of Embodiments 1 to 13, wherein the vector further includes one or more active substances.

Embodiment 15. The vector for delivering an active substance of Embodiment 14, wherein the vector further includes two or more active substances, wherein the active substances are the same or different.

Embodiment 16. The vector for delivering an active substance of Embodiment 14, wherein the active substance is included in the viral particle of the adeno-associated virus moiety.

Embodiment 17. The vector for delivering an active substance of any one of Embodiments 14 and 16, wherein the active substance is a transgene.

Embodiment 18. The vector for delivering an active substance of Embodiment 17, wherein the transgene is a gene associated with the treatment of neurological disorder.

Embodiment 19. The vector for delivering an active substance of Embodiment 17, wherein the gene associated with the treatment of neurological disorder is any one selected from MECP2, SCN1A, NF2, SNCA, LRRK2, APP, Tau, Nav1.7, C₉rof72, SOD1, DYRK1A, IT15, HTT, HEXA, RA11, PRGN, UBE3A, ABCA4, RP1, PAX6, USH2A, NRP1, SMN1, SMN2, GAA, PMP22, TTR, APOEε4, APOEε3, APOEε2, MAPT, GRN, AADC, GBA1, ASPA, CLN2, GLB1, SGSH, NAGLU, IDS, NPC1, SCA1(ATXN1), SCA3(ATXN3), SCA7(ATXN7), TARDBP(TDP-43), FRDA(FXN), SCN9A, SCN10A, and GAN.

Embodiment 20. The vector for delivering an active substance of any one of Embodiments 14 and 16, wherein the active substance is any one gene selected from a nucleic acid group associated with a CRISPR/Cas system and a nucleic acid group encoding a protein associated with a CRISPR/Cas system.

Embodiment 21. The vector for delivering an active substance of Embodiment 20, wherein the nucleic acid group associated with the CRISPR/Cas system includes crRNA, tracrRNA, and guideRNA, and the protein associated with the CRISPR/Cas system includes Cas9, Cas12, and Cas13.

Embodiment 22. A composition for delivering an active substance to a target site, including the vector for delivering an active substance of any one of Embodiments 1 to 21.

Embodiment 23. The composition for delivering an active substance of Embodiment 22, wherein the target site is the central nervous system, the peripheral nervous system, or nerve cells.

Embodiment 24. The composition for delivering an active substance of Embodiment 22, wherein the target site is a brain.

Embodiment 25. The composition for delivering an active substance of Embodiment 22, wherein the target site is a deep brain.

Embodiment 26. The composition for delivering an active substance of Embodiment 22, wherein the target site is a hippocampus, striatum, or olfactory bulb.

Embodiment 27. A pharmaceutical composition for treating a target disease, including the vector for delivering an active substance of any one of Embodiments 1 to 21.

Embodiment 28. The pharmaceutical composition for treating a target disease of Embodiment 27, wherein the target disease is a neurological disorder.

Embodiment 29. The pharmaceutical composition for treating a target disease of Embodiment 27, wherein the target disease is a central nervous system disorder.

Embodiment 30. The pharmaceutical composition for treating a target disease of Embodiment 29, wherein the central nervous system disorder is any one or more selected from a vascular disorder, a disorder caused by infection, a structural disorder, a functional disorder, a neurodegenerative disorder, a mood disorder, a neurodevelopmental disorder, and a psychiatric disorder.

Embodiment 31. The pharmaceutical composition for treating a target disease of Embodiment 27, wherein the target disease is a peripheral nervous system disorder.

Embodiment 32. The pharmaceutical composition for treating a target disease of Embodiment 27, wherein the target disease is a lysosomal storage disorder.

Embodiment 33. The pharmaceutical composition for treating a target disease of any one of Embodiments 27 to 32, the pharmaceutical composition further includes a pharmaceutically acceptable additive (excipient).

Embodiment 34. The pharmaceutical composition for treating a target disease of any one of Embodiments 27 to 32, The pharmaceutical composition further includes a therapeutic agent.

Embodiment 35. A method for treating a neurological disorder in a subject, the method including administering the vector for delivering an active substance of any one of Embodiments 1 to 21 into the cerebrospinal fluid of the subject.

Embodiment 36. The method for treating a neurological disorder in a subject of Embodiment 35, wherein the administration of the vector for delivering an active substance into the cerebrospinal fluid includes intrathecally injecting or intracerebroventricularly injecting a composition including the vector for delivering an active substance of any one of Embodiments 1 to 21 into a subject.

Embodiment 37. The method for treating a neurological disorder in a subject of any one of Embodiments 35 and 36, wherein the neurological disorder is a central nervous system disorder.

Embodiment 38. The method for treating a neurological disorder in a subject of any one of Embodiments 35 and 36, wherein the neurological disorder is a peripheral nervous system disorder.

Embodiment 39. The method for treating a neurological disorder in a subject of any one of Embodiments 35 and 36, wherein the neurological disorder is a lysosomal storage disorder.

Embodiment 40. A method for preparing the vector for delivering an active substance of Embodiment 1, the method including contacting a TTR ligand and an adeno-associated virus.

Embodiment 41. A method for preparing the vector for delivering an active substance of Embodiment 12, the method including contacting a TTR ligand represented by Formula 2-10 and an adeno-associated virus.

Embodiment 42. A method for preparing the vector for delivering an active substance of Embodiment 12, the method including mixing a first composition including a TTR ligand represented by Formula 2-10 and a second composition including an adeno-associated virus.

Embodiment 43. The method for preparing the vector for delivering an active substance of Embodiment 42, wherein a ratio of the number of moles of TTR ligand included in the first composition and the viral genome titer of adeno-associated virus included in the second composition is about 50:1, 100:1, 1000:1, 10000:1, or 100000:1.

Embodiment 44. The method for preparing the vector for delivering an active substance of Embodiment 42, wherein the TTR ligand is prepared by a method including:
(i) producing N-acetyl thyroxin by contacting T4 and N-acetyl hydroxysuccinimide; and
(ii) contacting N-acetyl thyroxin and N-hydroxy succinimide.

### EXAMPLES

### Culture of Cells

AAV293, HEK293T, Oli-neu, RT4-D6P2T, and SIM-A9 were used in the experiments of the present application. AAV293, HEK293T and RT4-D6P2T cells were cultured in Dulbecco's modified Eagle's medium (DMEM; Corning Cellgro) including 10% fetal bovine serum (FBS; Corning), 1% penicillin-streptomycin (pen-strep; Thermo Fisher Scientific, Waltham, MA) under 37° C. and 5% C₀₂ conditions. Oli-neu cells were cultured for at least 2-3 days at 37° C. in a SATO medium including 1% horse serum, gentamicin, and 1 mM dibutyryl cAMP (dbcAMP; Sigma) for differentiation. The cells were inoculated into a culture flask coated with 0.01% poly-L-lysine (PLL, Sigma). SIM-A9 cells were cultured at 37° C. and 5% CO₂ in DMEM/F12 medium including 5% horse serum and 10% fetal bovine serum.

### Production of AAV Vector

An AAV vector carrying cDNA encoding green fluorescent protein (GFP) or luciferase (hereinafter referred to as an AAV vector including a GFP gene or an AAV vector including a Luc gene) through a cytomegalovirus promoter was packaged into AAV293 cells using the calcium phosphate transient transfection method. The cells were harvested 48 hours after transfection, and then a freeze-thaw procedure was performed to form a lysate. The lysed solution including the viral vector was treated with benzonase nuclease (Sigma-Aldrich) to remove cellular genome fragments. After debris was removed, the viral vector was purified by an iodixanol density gradient method (OptiPrep™) using ultracentrifugation (360,000 g force; Type 100Ti rotor, Beckman Coulter) at 18° C. for 2 hours. A buffer of the AAV solution was exchanged using 1×PBS-0.01% (v/v) Tween-20 (Sigma-Aldrich) and using an Amicon ultra-15 centrifugal filter tube (10,000 MWCO, Millipore). The genome titer of the purified viral vector solution was determined using quantitative PCR (qPCR). The AAV solution was treated with a deoxyribonuclease (DNase I; Thermo Fisher Scientific) in a water bath at 37° C. for 30 minutes. The solution was then mixed with a proteinase (proteinase K; Thermo Fisher Scientific) at 37° C. for 90 minutes. A SYBR green master mix (Thermo Fisher Scientific) was used to perform quantitative PCR (qPCR, mini Opticon, Bio-Rad).

### Example 1. Synthesis of TTR Ligand

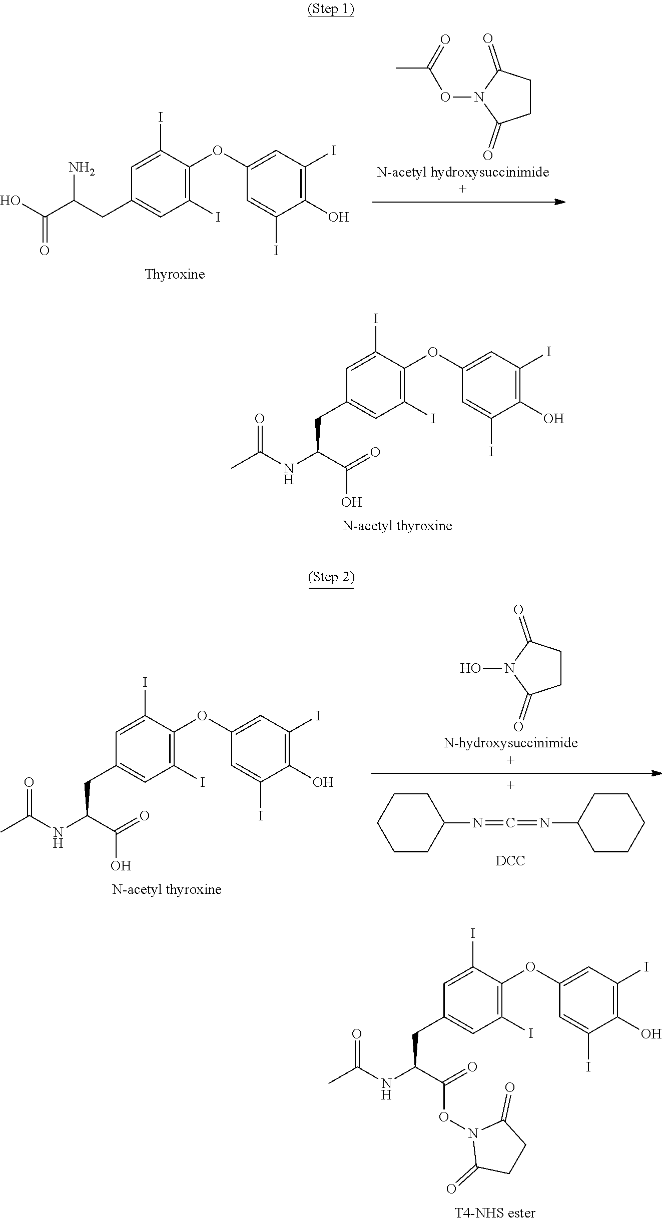

The TTR ligand disclosed in Example 1 is T4-NHS ester, which is disclosed as Formula 2-10 of the present application.

The inventors of the present application synthesized T4-NHS ester by the following method.

Step 1. After 0.1 g of L-thyroxine sodium salt in 20 mL of ethanol was reacted with 40 mg of N-acetyl hydroxysuccinimide and stirred at room temperature for 23 hours, the resulting product was evaporated in vacua and purified to obtain N-acetyl thyroxine.

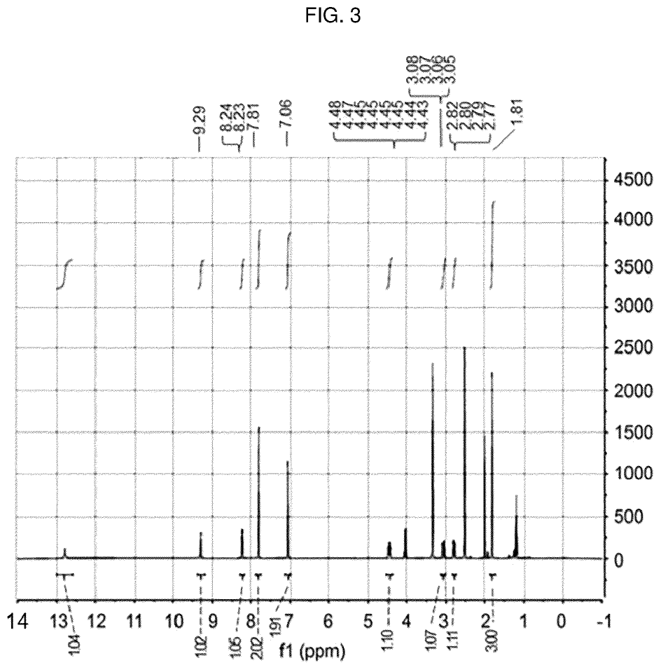

The H NMR data of N-acetyl thyroxine is shown in FIG. 3.

Step 2. After 1.65 mg of N-acetyl thyroxine synthesized in Step 1 and 0.32 mg of N-hydroxy succinimide were dissolved in 300 μl of dioxane, the resulting solution was cooled to 000. After cooling to 0° C. was completed, 0.45 mg of DCC in 20 μl of dioxane was added thereto, and the resulting mixture was stirred at room temperature for 24 hours to synthesize T4-NHS ester.

It was confirmed through H NMR that T4-NHS ester had been prepared.

In the present Example 1, for the following reasons, N-acetyl thyroxine was first synthesized by capping the amine group of T4 with a ketone group (Step 1), and the carboxyl group of N-acetyl thyroxine was substituted with NHS to synthesize T4-NHS ester (Step 2).

(i) When the amine group of T4 is not capped with a ketone group, there is a possibility that the ester group (or carboxy group) of the synthesized TTR ligand will react with the unreacted and remaining amine group of T4, and in this case, this hinders the subsequent production of the vector for delivering an active substance.

(ii) When the amine group of T4 is not capped with a ketone group and the carboxyl group is directly substituted with an ester group to produce a TTR ligand, there is a problem in that a TTR ligand produced from T4 is not sufficiently dissolved in the solvent used to produce T4-AAV. In addition, in this case, the TTR ligand produced from T4 may have the property of a zwitterion, which has a negative impact on the subsequent reaction between the TTR ligand and the adeno-associated virus.

### Example 2. Synthesis of Vector in which TTR Ligands are Conjugated

A vector in which TTR ligands are conjugated and which is synthesized in Example 2 is disclosed as Formula 1-5 of the present application.

### 2.1. Preparation of Sample 1 (T4-AAV9, Viral Genome Titer of AAV9 Mole of T4-NHS Ester=1:50)

Only dicyclohexylurea, which is a precipitate, was removed from the final product (composition including the synthesized T4-NHS ester of Formula 2-10) prepared through the process of Example 1 through a 0.45-μm filter. Assuming that the yield of T4-NHS ester was 70%, a reaction was performed by setting the ratio of the viral genome titer of AAV9 to the number of moles of T4-NHS ester to 1:50. In this case, by calculating the molecular weight, 7.5×10⁻⁶ μg of T4-NHS ester per 1×10⁹ vg/μL of AAV9 was added to prepare reactants including AAV9 and T4-NHS ester. The reactants including AAV9 and T4-NHS ester were mixed at a molar ratio of 1:50, and then stirred at 4° C. in a shaded state (protected from light) for 13 hours. After 13 hours of reaction, a reaction was performed at 0° C. for 10 minutes by mixing the reactants and an ammonium sulfate solution at a ratio of 3:1, and centrifugation was performed at 4° C. under the condition of 9000 g for 10 minutes. Thereafter, the supernatant including AAV9 and T4-NHS ester was transferred from the centrifuged tube to another tube. After addition of ammonium sulfate solution in an amount to ⅔ of the volume of the reactant to the tube including the corresponding supernatant, a reaction was performed at 0° C. for 20 minutes, and centrifugation was performed at 4° C. under the condition of 9000 g for 20 minutes. The precipitate generated by centrifugation was suspended in phosphate-buffered saline with Tween20 (PBS-TW), and then purified to obtain concentrated T4-AAV9.

### 2.2. Preparation of Sample 2 (T4-AAV9, Viral Genome Titer of AAV9 Mole of T4-NHS Ester=1:100)

Sample 2 was produced similarly to T4-AAV9 of Sample 1. However, in this case, a reaction was performed by setting the ratio of viral genome titer of AAV9 to the number of moles of T4-NHS ester to 1:100. Therefore, 1.5×10⁻⁵ μg of T4-NHS ester per 1×10⁹ vg/μL of AAV9 was added to prepare reactants including AAV9 and T4-NHS ester.

### 2.3. Preparation of Sample 3 (T4-AAV9, Viral Genome Titer of AAV9 Mole of T4-NHS Ester=1:1000)

Sample 3 was produced similarly to T4-AAV9 of Sample 1. However, in this case, a reaction was performed by setting the ratio of viral genome titer of AAV9 to the number of moles of T4-NHS ester to 1:1000. Therefore, 1.5×10⁻⁴ μg of T4-NHS ester per 1×10⁹ vg/μL of AAV9 was added to prepare reactants including AAV9 and T4-NHS ester.

### 2.4. Preparation of Sample 4 (T4-AAV9, Viral Genome Titer of AAV9 Mole of T4-NHS Ester=1:10000)

Sample 4 was produced similarly to T4-AAV9 of Sample 1. However, in this case, a reaction was performed by setting the ratio of viral genome titer of AAV9 to the number of moles of T4-NHS ester to 1:10000. Therefore, 1.5×10⁻³ μg of T4-NHS ester per 1×10⁹ vg/μL of AAV9 was added to prepare reactants including AAV9 and T4-NHS ester.

### 2.5. Preparation of Sample 5 (T4-AAV9, Viral Genome Titer of AAV9 Mole of T4-NHS Ester=1:100000)

Sample 5 was produced similarly to T4-AAV9 of Sample 1. However, in this case, a reaction was performed by setting the ratio of viral genome titer of AAV9 to the number of moles of T4-NHS ester to 1:100000. Therefore, 1.5×10⁻² μg of T4-NHS ester per 1×10⁹ vg/μL of AAV9 was added to prepare reactants including AAV9 and T4-NHS ester.

### 2.6. Preparation of Sample 6 (T4-AAV2)

T4-AAV2 of Sample 6 was produced by the following method.

Step 1. After 0.2 g of L-thyroxine sodium salt in 25 mL of ethanol was reacted with 60 mg of N-acetyl hydroxysuccinimide and stirred at room temperature for 23 hours, the resulting product was evaporated in vacuo and purified to obtain N-acetyl thyroxine.

Step 2. After 38 mg of N-acetyl thyroxine synthesized in Step 1 and 9 mg of N-hydroxy succinimide were dissolved in 300 μl of dioxane, the resulting solution was cooled to 0° C. After cooling to 0° C. was completed, 4 mg of DCC in 200 μl of dioxane was added, and the resulting mixture was stirred at room temperature for 26 hours, evaporated in vacuo, extracted with ethyl acetate, and then dried to obtain T4-NHS ester.

AAV2 in PBS was added to a final product produced through the process of Step 2 dissolved in a 2% DMSO solution. A reaction was performed by setting the molar ratio of AAV:T4-NHS ester to 1:100000. In this case, by calculating the molecular weight, 1.5×10⁻² μg of T4-NHS ester per 1×10⁹ vg/μL of AAV2 was added to prepare reactants including AAV2 and T4-NHS ester. Thereafter, the reactants were stirred at 4° C. in a shaded state (protected from light) for 12 hours. After 12 hours of reaction, the reaction product was transferred to a dialysis cassette (Piece, 10 kD cutoff) and dialyzed with acetone:PBS (1:1) for 2 hours, and the buffer was exchanged three times with PBS to obtain purified T4-AAV2.

### 2.7. Preparation of Sample 7 (T4-AAVr3.45)

T4-AAVr3.45 of Sample 7 was produced by the following method.

Step 1. After 0.2 g of L-thyroxine sodium salt in 25 mL of ethanol was reacted with 60 mg of N-acetyl hydroxysuccinimide and stirred at room temperature for 23 hours, the resulting product was evaporated in vacuo and purified to obtain N-acetyl thyroxine.

Step 2. After 38 mg of N-acetyl thyroxine synthesized in Step 1 and 9 mg of N-hydroxy succinimide were dissolved in 300 μl of dioxane, the resulting solution was cooled to 0° C. After cooling to 0° C. was completed, 4 mg of DCC in 200 μl of dioxane was added, and the resulting mixture was stirred at room temperature for 26 hours, evaporated in vacuo, extracted with ethyl acetate, and then dried to obtain T4-NHS ester.

AAVr3.45 in PBS was added to a final product produced through the process of Step 2 dissolved in a 2% DMSO solution. A reaction was performed by setting the molar ratio of AAV:T4-NHS ester to 1:100000. In this case, by calculating the molecular weight, 1.5×10⁻² μg of T4-NHS ester per 1×10⁹ vg/μL of AAVr3.45 was added to prepare reactants including AAVr3.45 and T4-NHS ester. Thereafter, the reactants were stirred at 4° C. in a shaded state (protected from light) for 12 hours. After 12 hours of reaction, the reaction product was transferred to a dialysis cassette (Piece, 10 kD cutoff) and dialyzed with acetone:PBS (1:1) for 2 hours, and the buffer was exchanged three times with PBS to obtain purified T4-AAVr3.45.

### Example 3. Confirmation of Synthesis of Vector in which TTR Ligands are Conjugated

### 3.1. Confirmation of Synthesis of T4-AAV9 of Sample 1

The production of T4-AAV9 of Sample 1 is confirmed.

The zeta potential of T4-AAV9 of Sample 1 is measured to confirm the production of T4-AAV9 of Sample 1. In order to confirm the production of T4-AAV9 of Sample 1, the zeta potential of T4-AAV9 of Sample 1 is measured using an ELS-1000ZS apparatus manufactured by Otsuka Electronics.

The production of T4-AAV9 of Sample 1 is confirmed by checking whether a reaction between T4-AAV9 of Sample 1 and an anti-T4 antibody occurred.

### 3.2. Confirmation of Synthesis of T4-AAV9 of Sample 2

The inventors of the present application confirmed that a vector in which TTR ligands are conjugated had been produced. The inventors of the present application confirmed that T4-AAV9 of Sample 2 was produced by measuring the zeta potential of T4-AAV9 of Sample 2.

To confirm the production of T4-AAV9 of Sample 2, the zeta potential was measured using an ELS-1000ZS apparatus manufactured by Otsuka Electronics. Measurements were performed after diluting AAV9 including a GFP gene and AAV9-T4 including a GFP gene to a concentration of 1×10⁸ vg/ml using PBS.

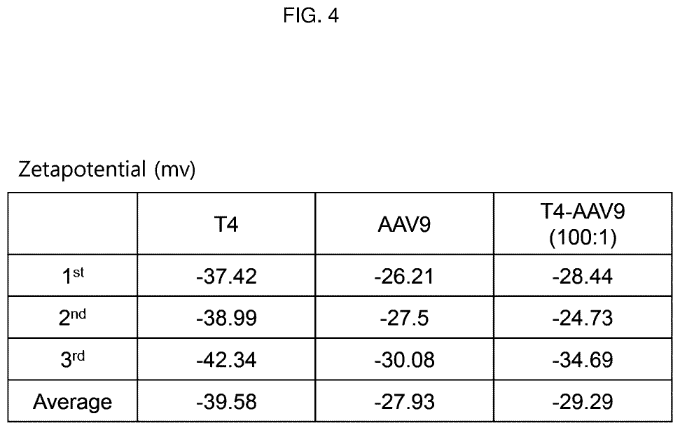

The results of measuring the zeta potential of T4-AAV9 of Sample 2 are shown in FIG. 4. In FIG. 4, a group denoted as T4-AAV9 (100:1) is the result of measuring the zeta potential of T4-AAV9 of Sample 2. As shown in FIG. 4, there is a difference in the zeta potential of AAV9 and T4-AAV9 of Sample 2, indicating that T4-AAV9 was successfully synthesized.

The production of T4-AAV9 of Sample 1 is confirmed by checking whether a reaction between T4-AAV9 of Sample 1 and an anti-T4 antibody is occurred.

### 3.3. Confirmation of Synthesis of T4-AAV9 in Sample 3

The production of T4-AAV9 of Sample 3 is confirmed.

The zeta potential of T4-AAV9 of Sample 3 is measured to confirm the production of T4-AAV9 of Sample 3. In order to confirm the production of T4-AAV9 of Sample 3, the zeta potential of T4-AAV9 of Sample 3 is measured using an ELS-1000ZS apparatus manufactured by Otsuka Electronics.

The production of T4-AAV9 of Sample 3 is confirmed by checking whether a reaction between T4-AAV9 of Sample 3 and an anti-T4 antibody is occurred.

### 3.4. Confirmation of Synthesis of T4-AAV9 in Sample 4

The production of T4-AAV9 of Sample 4 is confirmed.

The zeta potential of T4-AAV9 of Sample 4 is measured to confirm the production of T4-AAV9 of Sample 4. In order to confirm the production of T4-AAV9 of Sample 4, the zeta potential of T4-AAV9 of Sample 4 is measured using an ELS-1000ZS apparatus manufactured by Otsuka Electronics.

The production of T4-AAV9 of Sample 4 is confirmed by checking whether a reaction between T4-AAV9 of Sample 4 and an anti-T4 antibody occurred.

### 3.5. Confirmation of Synthesis of T4-AAV9 in Sample 5

The production of T4-AAV9 of Sample 5 is confirmed.

The zeta potential of T4-AAV9 of Sample 5 is measured to confirm the production of T4-AAV9 of Sample 5. In order to confirm the production of T4-AAV9 of Sample 5, the zeta potential of T4-AAV9 of Sample 5 is measured using an ELS-1000ZS apparatus manufactured by Otsuka Electronics.

The production of T4-AAV9 of Sample 4 is confirmed by checking whether a reaction between T4-AAV9 of Sample 5 and an anti-T4 antibody is occurred.

3.6. Confirmation of synthesis of T4-AAV2 in Sample 6

The inventors of the present application confirmed that a vector in which TTR ligands are conjugated had been produced. The inventors of the present application confirmed that T4-AAV2 was produced by measuring the zeta potential of T4-AAV2.

The zeta potentials of T4-AAV2 and T4-unconjugated AAV2 were measured using a dynamic light scattering (DLS) particle size analyzer (Zen3600, Malvern Instruments, Worcestershire, United Kingdom).

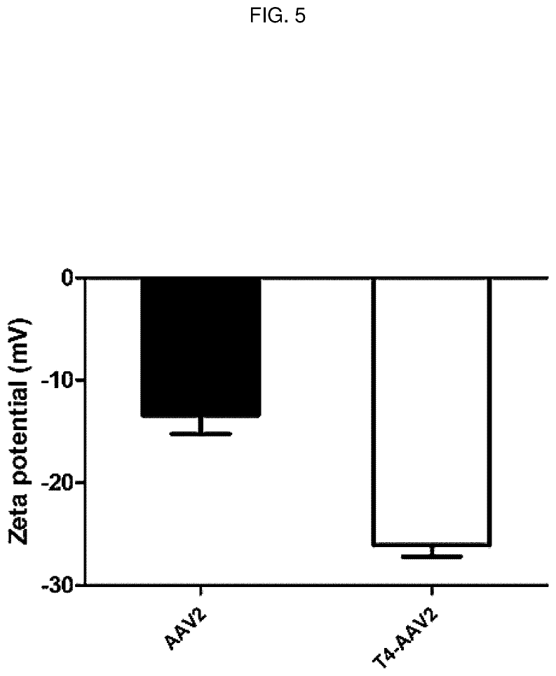

The results of measuring the zeta potential of T4-AAV2 are shown in FIG. 5. As shown in FIG. 5, there is a difference in the zeta potential of AAV2 and T4-AAV2, indicating that T4-AAV2 was successfully synthesized.

### 3.7. Confirmation of Synthesis of T4-AAVr3.45 in Sample 7

The production of T4-AAVr3.45 of Sample 7 is confirmed.

The zeta potential of T4-AAVr3.45 of Sample 7 is measured to confirm the production of T4-AAVr3.45 of Sample 7. In order to confirm the production of T4-AAVr3.45 of Sample 7, the zeta potential of T4-AAVr3.45 of Sample 7 is measured using an ELS-1000ZS apparatus manufactured by Otsuka Electronics.

The production of T4-AAVr3.45 of Sample 7 is confirmed by checking whether a reaction between T4-AAVr3.45 of Sample 7 and an anti-T4 antibody is occurred.

### Example 4. Confirmation of Transduction Ability of Vector in which TTR Ligands are Conjugated (In Vitro)

### 4.1. Confirmation of Transduction Ability of T4-AAV9 (In Vitro)

The inventors of the present application confirmed the transduction ability of T4-AAV9. The T4-AAV9 transduction ability of Sample 2, Sample 3, Sample 4, and Sample 5 were confirmed. In this case, the GFP gene was included in the vector of each sample for processing.

HEK293T cells were seeded at 2×10⁴ cells into each well of a 48-well plate, and then incubated at 37° C. After 16 hours, each sample was infected at a multiplicity of infection (MOI) of 3×10⁵.

The medium was replaced 12 hours after infection and the cells were incubated at 37° C. 48 hours after infection, the degree of GFP expression was confirmed using a fluorescence microscope, and the percentage of GFP-positive cells was confirmed using a flow cytometer.

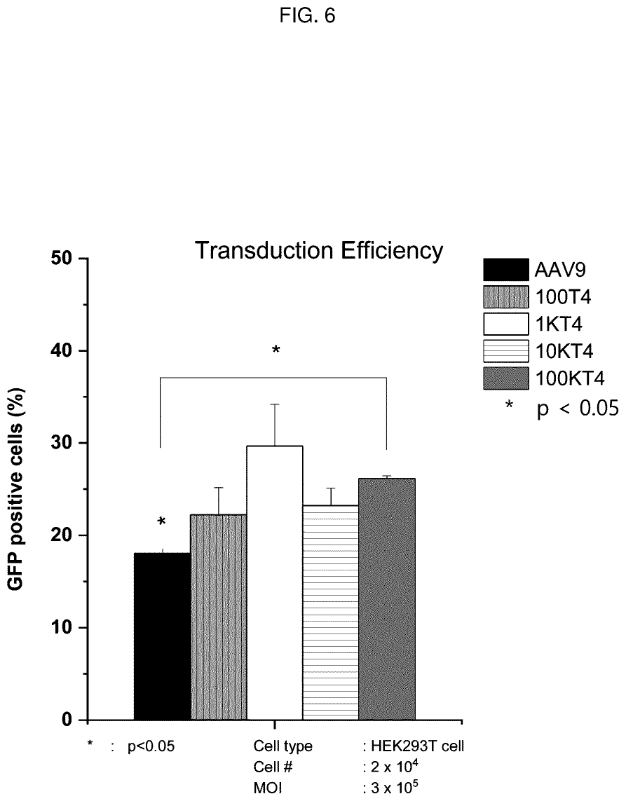

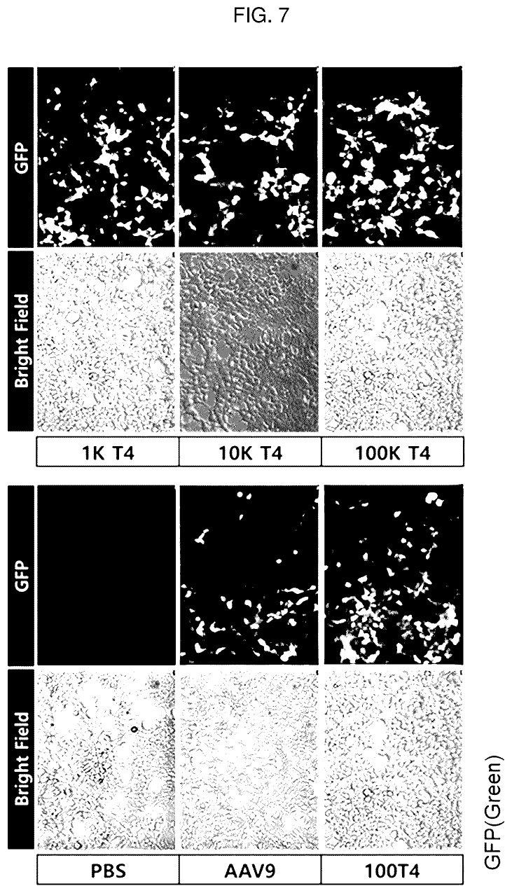

The experimental results are shown in FIGS. 6 and 7. In FIGS. 6 and 7, the AAV9 group means a group treated with AAV9 in which T4 is not conjugated, the 100T4 group means a group treated with T4-AAV9 of Sample 2, the 1K T4 group means a group treated with T4-AAV9 of Sample 3, the 10K T4 group means a group treated with T4-AAV9 of Sample 4, and the 100K T4 group means a group treated with T4-AAV9 of Sample 5. In the HEK293T cell infection results, it was confirmed that the GFP expression rate was higher in the T4-AAV9 groups (groups treated with Sample 2, Sample 3, Sample 4, and Sample 5) than in a comparison group AAV9 (group treated with only AAV9 including the GFP gene). In addition, it was confirmed that there was no large difference in the GFP expression rate between the groups treated with T4-AAV9. This is interpreted that the conjugation of T4 does not negatively impact the transduction ability of AAV9.

4.2. Confirmation of Transduction Ability of T4-AAV2 of Sample 6 (in vitro)

The inventors of the present application confirmed the transduction ability of T4-AAV2 of Sample 6. In this case, the GFP gene was included in the vector for processing.

HEK293T cells with a specified cell density (2×10⁴ cells/200 μl) were cultured in a 48-well culture plate for 12 hours. Thereafter, for each condition, infection was performed at MOIs of 1000, 5000, and 10000. Transduction results were analyzed 48 hours after infection. Cells were analyzed using a fluorescence-activated cell sorter at the Medical Research Center of Yonsei University College of Medicine (LSR II, Beckman Coulter).

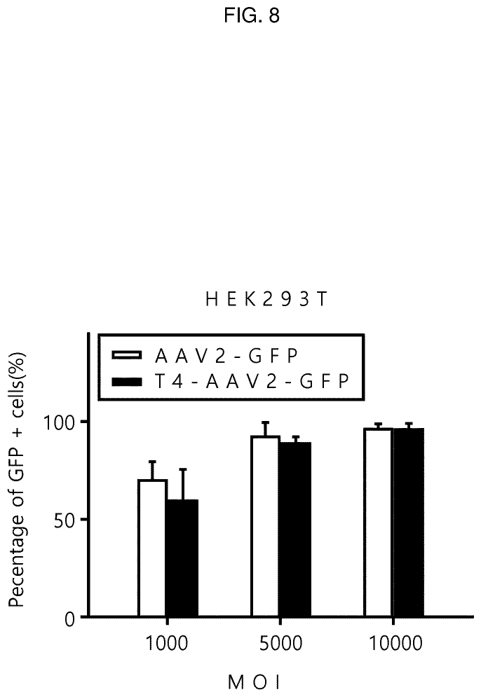

The experimental results are shown in FIG. 8. In FIG. 8, the group denoted as AAV2-GFP means a group treated with AAV2 including GFP gene, in which a TTR ligand is not conjugated and which includes, and the group denoted as T4-AAV2-GFP means a group treated with T4-AAV2 of Sample 6 including the GFP gene. In the HEK293T cell infection results, it was confirmed that there was no difference in transduction ability between a comparison group AAV2 (a group treated with only AAV2 including the GFP gene) and the T4-AAV2 group (a group treated with Sample 6).

### 4.3. Confirmation of Transduction Ability of T4-AAVr3.45 of Sample 7 (In Vitro)

The inventors of the present application confirmed the transduction ability of T4-AAVr3.45 of Sample 7. In this case, the GFP gene was included in the vector.

RT4-D6P2T cells were cultured in a culture plate for 12 hours. Thereafter, for each condition, infection was performed at MOIs of 1000, 5000, and 10000. Transduction results were analyzed 48 hours after infection.

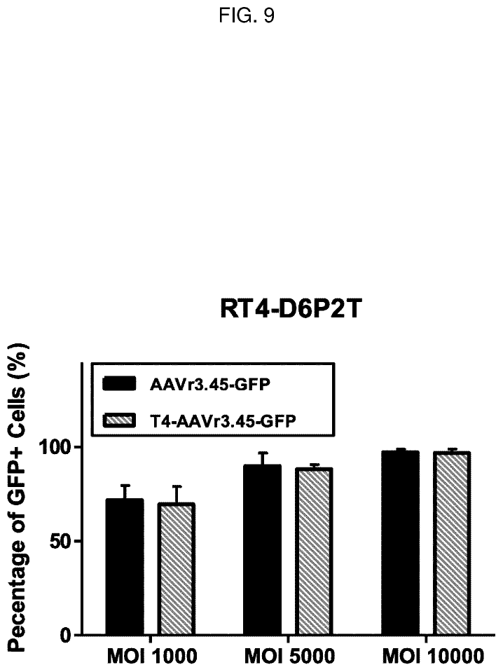

The experimental results are shown in FIG. 9. In FIG. 9, the group denoted as AAVr3.45-GFP means a group treated with AAVr3.45 including the GFP gene, in which a TTR ligand is not conjugated, and the group denoted as T4-AAVr3.45-GFP means a group treated with T4-AAVr3.45 of Sample 7 including the GFP gene. In the RT4-D6P2T infection results, it was confirmed that there was no difference in transduction ability between the AAVr3.45 group (a group treated with only AAV2 including the GFP gene), which is a comparison group, and the T4-AAVr3.45 group (a group treated with Sample 7).

### Example 5. Confirmation of Efficacy for Delivering Active Substance to Target Site of Vector in which TTR Ligands are Conjugated 1 (Luciferase)

The inventors of the present application confirmed the efficacy for transferring an active substance to a target site (for example, the brain) of the vector in which TTR ligand are conjugated by administering the vector into cerebrospinal fluid.

Furthermore, the inventors of the present application confirmed whether the vectors in which TTR ligand are conjugated administered into the cerebrospinal fluid are accumulated in the liver.

### 5.1. Confirmation of Delivering Efficacy of Active Substance of T4-AAV9 to Central Nervous System.

The inventors of the present application confirmed the delivering efficacy of the active substance of the vector in which TTR ligands are conjugated to the central nervous system. The efficacies for delivering the active substance to central nervous system of T4-AAV9 of Sample 1 and T4-AAV9 of Sample 2 were confirmed. In this case, the Luc gene was included in the vector, and after the active substance was delivered, fluorescence generated from luciferase and the fluorescent material was observed at the target site (brain).

The inventors of the present application confirmed the efficacy for delivering the active substance to the central nervous system of the vector in which TTR ligands are conjugated by the following method.

After 6-week-old mice were acclimatized for one week, animal experiments were performed on 7-week-old mice. The T4-AAV9 vector including the GFP gene was administered at 4×10⁹ vg in a total volume of 2 μL per mouse, and the T4-AAV9 vector including the Luc gene was administered at 4×10¹⁰ vg in a total volume of 2 μL per mouse. In this case, each vector was administered into the cerebrospinal fluid of mice through intracerebroventricular injection (ICV). For mice administered T4-AAV9 including the Luc gene, in vivo imaging was performed using an IVIS apparatus after respiratory anesthesia on days 4, 8, 12, 16, and 20 after administration. The mice were dissected for ex vivo imaging on day 21. Ex vivo imaging was performed using an IVIS apparatus.

Mice administered T4-AAV9 including the GFP gene were dissected for ex vivo imaging on day 21 after administration. Furthermore, mice administered T4-AAV9 including the Luc gene were also dissected for ex vivo imaging on day 21. Ex vivo imaging was performed using an IVIS apparatus.

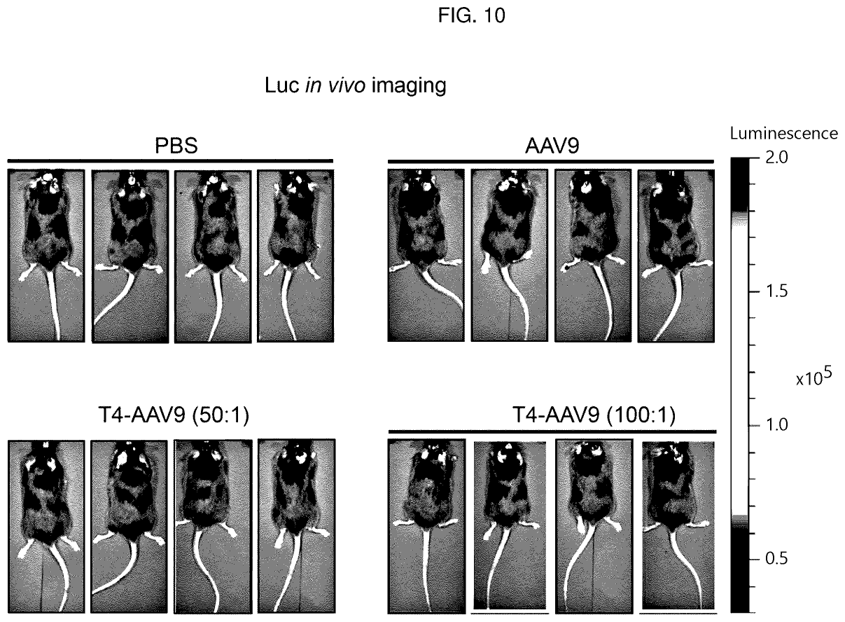

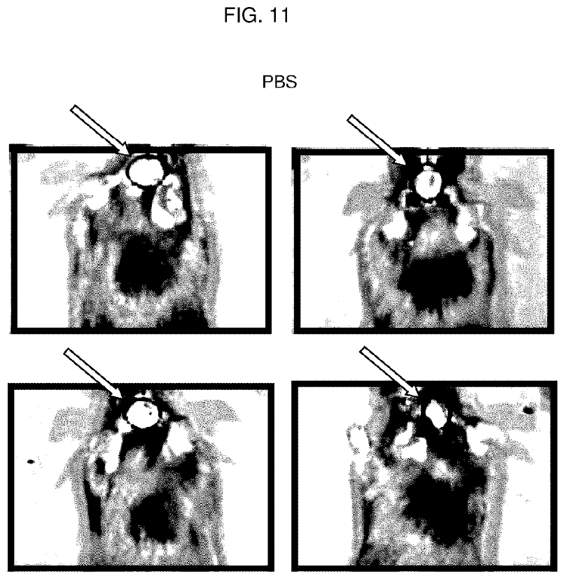

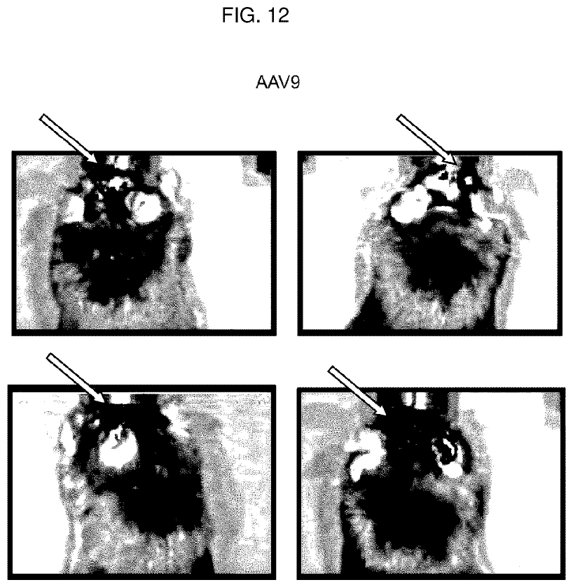

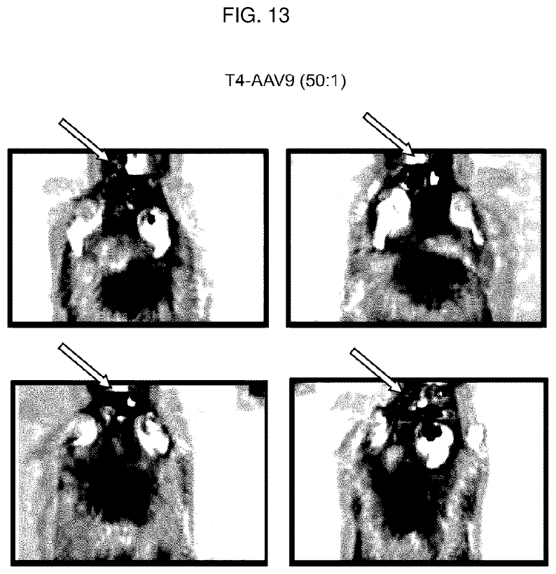

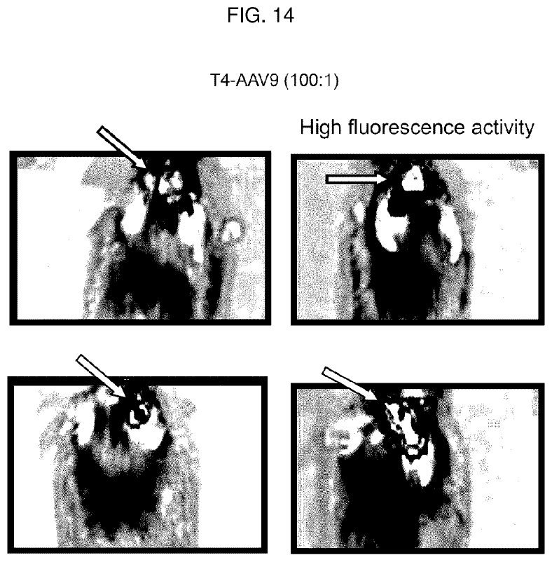

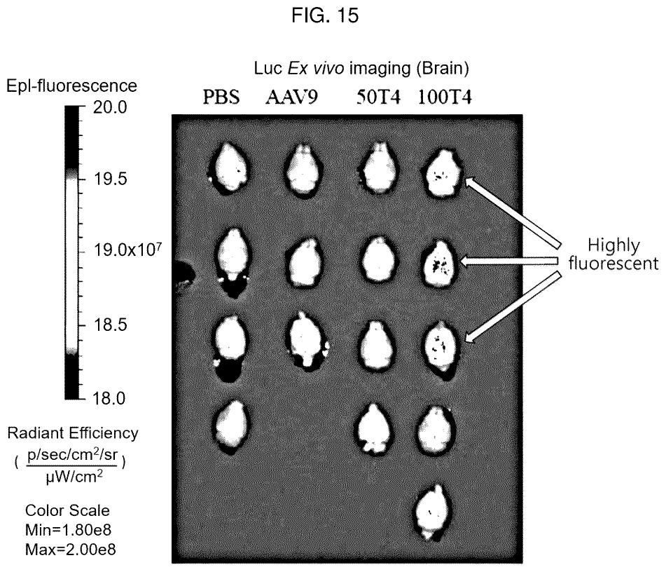

The in vivo imaging results of measuring fluorescence in the brain are shown in FIGS. 10 to 14, and the ex vivo imaging results are shown in FIG. 15. Among them, FIGS. 11 to 14 are enlarged views of the results in FIG. 10 for each group. In FIGS. 10 to 14, the group denoted as T4-AAV9 (50:1) is a group treated with T4-AAV9 of Sample 1, and the group denoted as T4-AAV9 (100:1) is a group treated with T4-AAV9 of Sample 2. In FIG. 15, the group denoted as 50T4 is a group treated with T4-AAV9 of Sample 1, and the group denoted as 100T4 is a group treated with T4-AAV9 of Sample 2.

As a result of administering the T4-AAV9 vectors of Sample 1 and Sample 2 into cerebrospinal fluid, more fluorescence was observed in the brain compared to the control PBS and the AAV9 sample. This indicates that more T4-AAV9 carrying the Luc gene reached the brain in Samples 1 and 2 compared to the control. Therefore, when using the vector in which TTR ligands are conjugated, the active substance can be successfully delivered to the central nervous system.

### 5.2. Confirmation of Accumulation of T4-AAV9 in Liver.

The inventors of the present application confirmed a degree of liver accumulation of the vector in which TTR ligands are conjugated. The more the vector accumulates in the liver, the less the ability to deliver the active substance to the target site. The inventors of the present application confirmed degree of liver accumulation of T4-AAV9 of Sample 1 and T4-AAV9 of Sample 2 administered into cerebrospinal fluid. In this case, the Luc gene was included in the vector, and fluorescence was measured in the liver.

The experimental method is the same as the method described in Example 5.1.

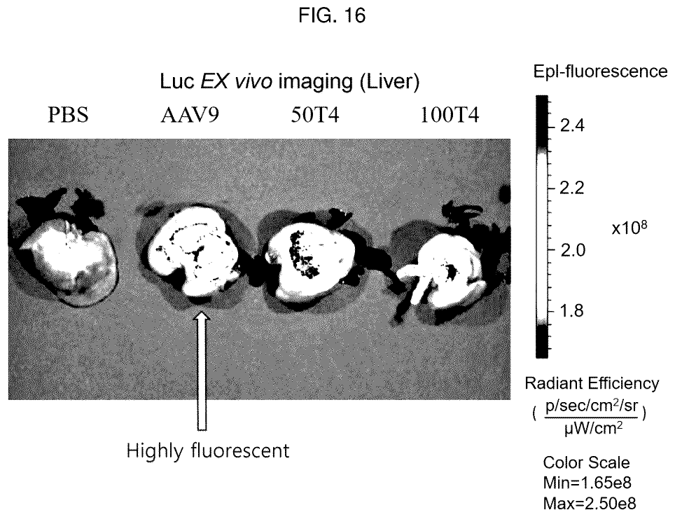

The results of an ex vivo imaging experiment measuring fluorescence in the liver are shown in FIG. 16. In FIG. 16, the group denoted as 50T4 is a group treated with T4-AAV9 of Sample 1, and the group denoted as 100T4 is a group treated with T4-AAV9 of Sample 2.

Fluorescence generated from luciferase and a fluorescent material was observed in the liver after administering T4-AAV9 from Samples 1 and 2 into cerebrospinal fluid. It was confirmed that the fluorescence observed in the liver in the experimental groups administered Sample 1 and Sample 2 was lower than the fluorescence observed in the control administered the AAV9 sample, which is one of the controls. This indicates that when T4-AAV9 is administered into cerebrospinal fluid, fewer vectors leak from the cerebrospinal fluid and accumulate in the liver than when AAV9 is administered.

Therefore, when using a vector in which a TTR ligand is conjugated, an active substance can be more efficiently delivered to the central nervous system.

### Example 6. Confirmation of Efficacy for Delivering Active Substance to Target Site of Vector in which TTR Ligands are Conjugated 2 (GFP)

### 6.1. Confirmation of Delivery Efficacy of T4-AAV9 to Deep Brain 1

The inventors of the present application confirmed the delivery efficacy to deep brain of the vector in which TTR ligands are conjugated, by administration the vectors into cerebrospinal fluid.

The inventors of the present application confirmed the delivery efficacy, to each of the olfactory bulb, striatum, hippocampus, and midbrain, of each of T4-AAV9 of Sample 1 and T4-AAV9 of Sample 2 including GFP gene, administered into cerebrospinal fluid.

T4-AAV9 including the GFP gene (T4-AAV9 expressing GFP) was intracerebroventricularly injected (ICV) into 2-month-old male C₅₇BL/6N mice. For the PBS group, the AAV9 group, and the T4-AAV9 groups (T4-AAV9 of Sample 1 and T4-AAV9 of Sample 2), administration was performed by intracerebroventricular injection, and in this case, each group was administered at specific coordinates (anteroposterior, −1.0 mm, mediolateral 0.6 mm, dorsoventral 2.0 mm) from the deep part of the mouse skull by a stereotaxic injection method. AAV9 and T4-AAV9 were each administered at 2×10⁹ vg/μl. At the time of injection, the injection rate was 0.4 μl/min, and the animals were anesthetized and perfused using cold PBS and 4% paraformaldehyde.

Brains were fixed with 4% paraformaldehyde and then stored in 30% sucrose until used for experiments. Thereafter, an experiment was performed using an immunohistochemical staining experimental method, and tissue samples (brain section cells) were reacted with the GFP antibody O/N at 4° C.

Thereafter, the tissue sample (brain section cells) was reacted at 25° C. for 1 hour using an appropriate fluorescent antibody (Alexa Fluor 488) or fluorescent primary antibody (Fluoro Myelin-Red/Alexa Fluor 647-tubulin beta 3).

After the reaction, the tissue sample was analyzed with a Carl Zeiss confocal microscope, and immunofluorescence images of the tissue sample were obtained.

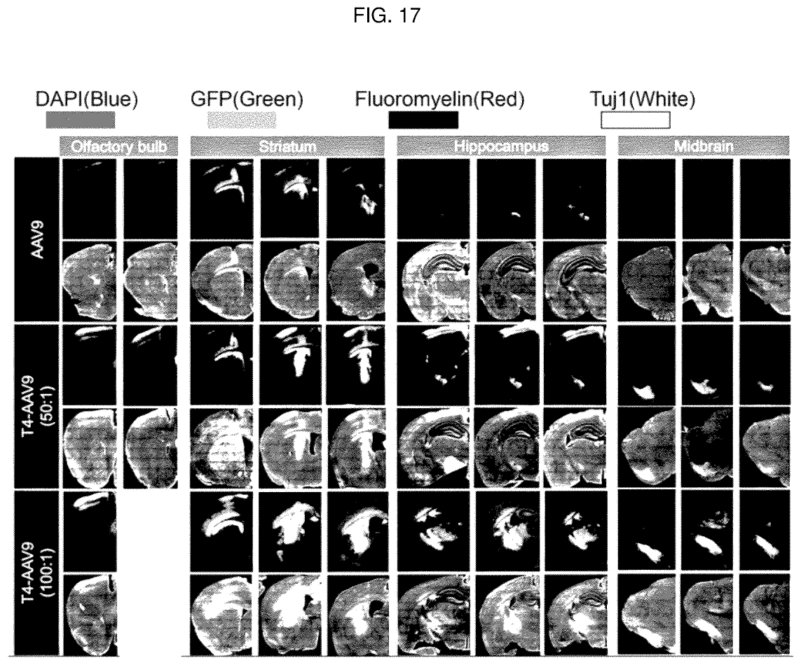

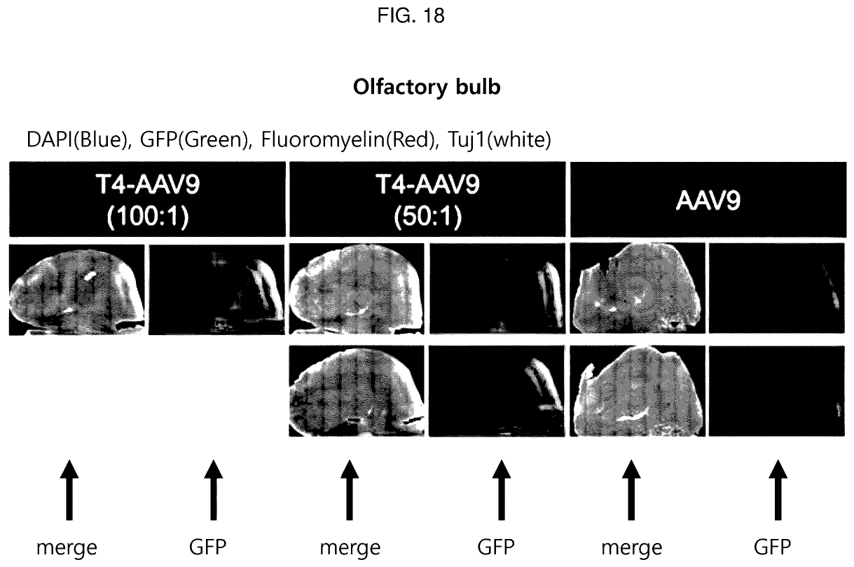

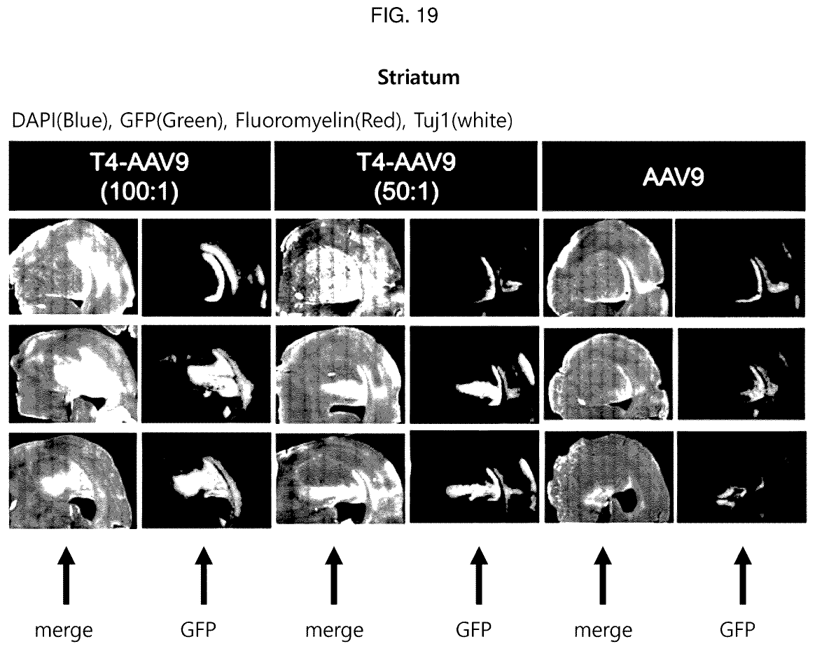

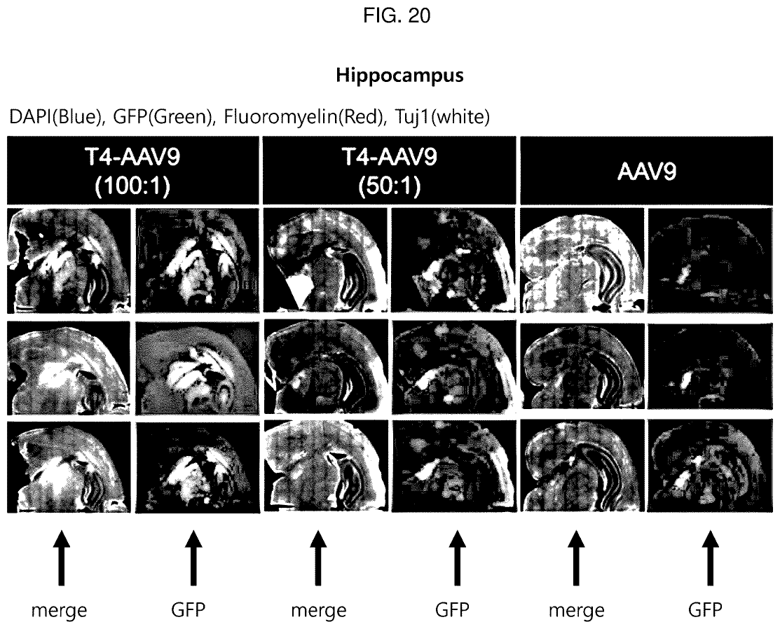

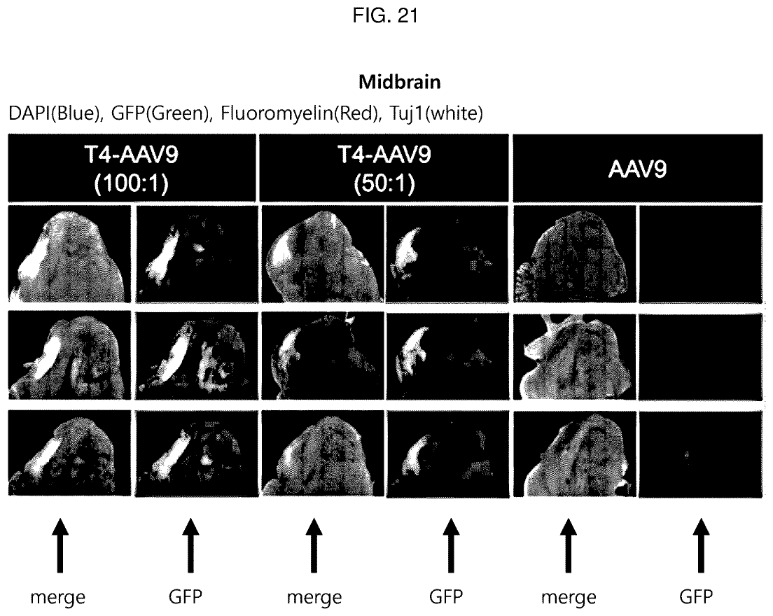

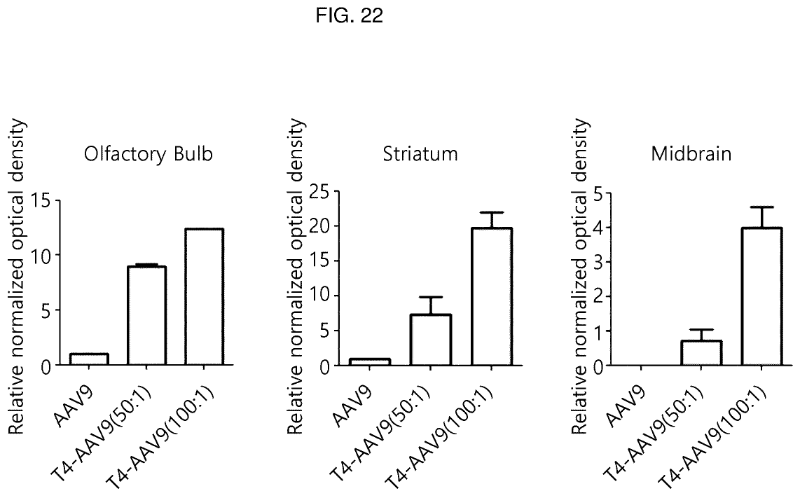

The experimental results are shown in FIGS. 17 to 21 and FIG. 22. Among them, FIGS. 18 to 21 show the results shown in FIG. 17 enlarged for each area (olfactory bulb, striatum, hippocampus, and midbrain) of the brain. In FIGS. 17 to 22, the group denoted as T4-AAV9 (50:1) means a group treated with T4-AAV9 of Sample 1, and the group denoted as T4-AAV9 (100:1) means a group treated with T4-AAV9 of Sample 2.

For each group, the intensity of fluorescence generated from GFP was confirmed in each area of the brain. It was confirmed that T4-AAV9 of Sample 1 and T4-AAV9 of Sample 2 have a higher efficacy of delivering the active substance to the olfactory bulb, striatum, hippocampus, and midbrain than AAV9, which is the comparison group.

Furthermore, it was confirmed that the T4-AAV9 of Sample 2 was better than the T4-AAV9 of Sample 1 in terms of the efficacy of delivering the active substance to the deep brain.

This indicates that the efficacy for delivering the active substance to central nervous system of the vector in which TTR ligands are conjugated is better than that of AAV in which the TTR ligand is not conjugated.

### 6.2. Confirmation of Delivery Efficacy to Deep Brain (Striatum, Ipsilateral) of T4-AAV9 2

The inventors of the present application confirmed the delivery efficacy to the deep brain of the vector in which TTR ligands are conjugated, administered into cerebrospinal fluid.

The inventors of the present application confirmed the delivery efficacy to the striatum (ipsilateral) of T4-AAV9 of Sample 1 and T4-AAV9 of Sample 2, which include the GFP gene, administered into cerebrospinal fluid.

Each of the PBS, AAV9, T4-AAV9 of Sample 1, and T4-AAV9 of Sample 2 was administered into the cerebrospinal fluid of mice in the same manner as the method described in Example 6.1.

Brains were fixed with 4% paraformaldehyde and then stored in 30% sucrose until used for experiments. Thereafter, an experiment was performed using an immunohistochemical staining experimental method, and tissue samples (brain section of the striatum) were reacted with the GFP antibody O/N at 4° C.

Thereafter, the tissue sample (brain section of the striatum) was reacted at 25° C. for 1 hour using an appropriate fluorescent antibody (Alexa Fluor 488) or fluorescent primary antibody (Fluoro Myelin-Red/Alexa Fluor 647-tubulin beta 3).

After the reaction, the tissue sample was analyzed with a Carl Zeiss confocal microscope, and immunofluorescence images of the tissue sample were obtained.

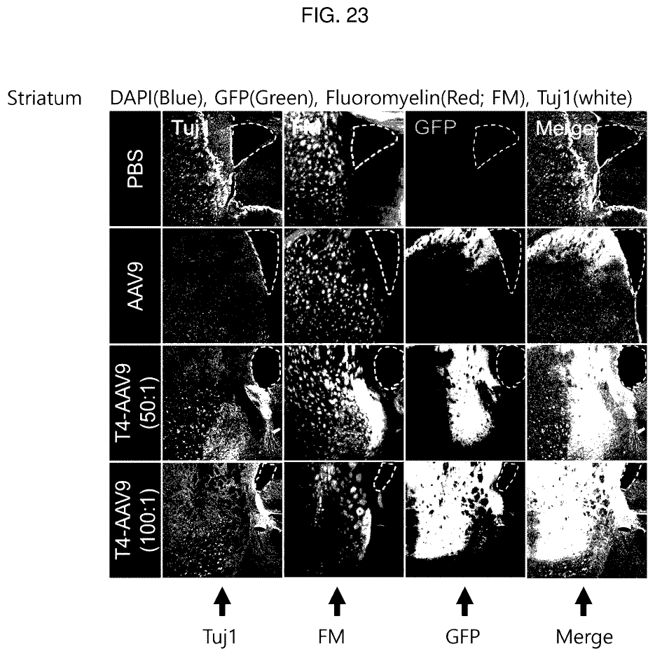

The experimental results are shown in FIG. 23. In FIG. 23, the group denoted as T4-AAV9 (50:1) means a group treated with T4-AAV9 of Sample 1, and the group denoted as T4-AAV9 (100:1) means a group treated with T4-AAV9 of Sample 2. In FIG. 23, the intensity of fluorescence generated from GFP in the striatum (ipsilateral) of the brain was observed for each condition. It was confirmed that the T4-AAV9 of Sample 1 and the T4-AAV9 of Sample 2 were better than the AAV9, which is the comparison group, in terms of the efficacy of delivering the active substance to the deep brain.

This indicates that the efficacy of delivering the active substance to central nervous system of the vector in which the TTR ligands are conjugated is better than that of AAV in which the TTR ligand is not conjugated.

### 6.3. Confirmation of Delivery Efficacy to Deep Brain (Hippocampus, Ipsilateral) of T4-AAV9 3

The inventors of the present application confirmed the delivery efficacy to deep brain of the vector in which the TTR ligands are conjugated, administered into cerebrospinal fluid.

The inventors of the present application confirmed the delivery efficacy to the hippocampus (ipsilateral) of T4-AAV9 of Sample 1 and T4-AAV9 of Sample 2, which include the GFP gene, administered into cerebrospinal fluid.

Each of the PBS, AAV9, T4-AAV9 of Sample 1, and T4-AAV9 of Sample 2 was administered into the cerebrospinal fluid of mice in the same manner as the method described in Example 6.1.

Brains were fixed with 4% paraformaldehyde and then stored in 30% sucrose until used for experiments. Thereafter, an experiment was performed using an immunohistochemical staining experimental method, and tissue samples (brain section of the hippocampus) were reacted with the GFP antibody O/N at 4° C.

Thereafter, the tissue sample (brain section of the hippocampus) was reacted at 25° C. for 1 hour using an appropriate fluorescent antibody (Alexa Fluor 488) or fluorescent primary antibody (Fluoro Myelin-Red/Alexa Fluor 647-tubulin beta 3).

After the reaction, the tissue sample was analyzed with a Carl Zeiss confocal microscope, and immunofluorescence images of the tissue sample were obtained.

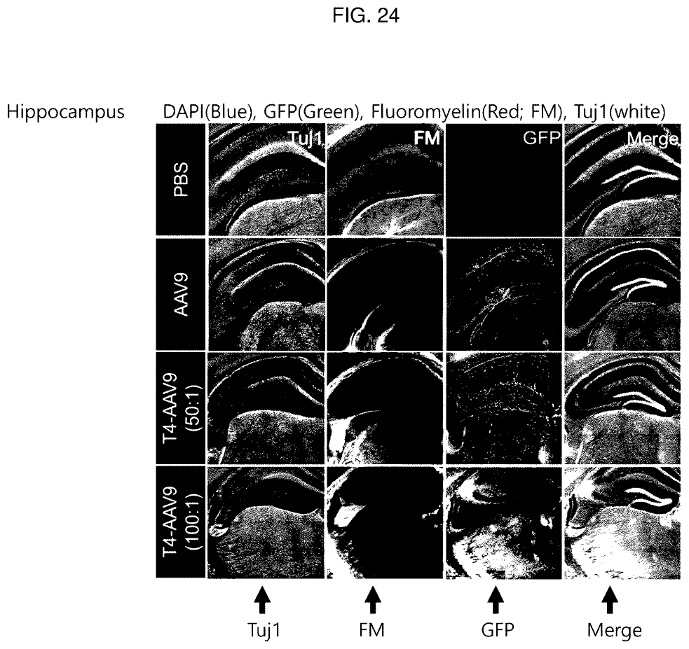

The experimental results are shown in FIG. 24. In FIG. 24, the group denoted as T4-AAV9 (50:1) means a group treated with T4-AAV9 of Sample 1, and the group denoted as T4-AAV9 (100:1) means a group treated with T4-AAV9 of Sample 2. As shown in FIG. 24, the intensity of fluorescence generated from GFP in the hippocampus (ipsilateral) of the brain was observed for each condition. It was confirmed that the T4-AAV9 of Sample 1 and the T4-AAV9 of Sample 2 were better than the AAV9, which is the comparison group, in terms of the efficacy of delivering the active substance to the deep brain.

This indicates that the efficacy of delivering the active substance to the central nervous system of the vector in which the TTR ligands are conjugated is better than that of AAV in which the TTR ligand is not conjugated.

### 6.4. Confirmation of Accumulation of T4-AAV9 in Liver

The inventors of the present application confirmed a degree of liver accumulation of the vector in which TTR ligands are conjugated. The more the vector accumulates in the liver, the less the ability to deliver the active substance to the target site. The inventors of the present application confirmed the degree of liver accumulation of T4-AAV9 of Sample 1 and T4-AAV9 of Sample 2 administered into cerebrospinal fluid. In this case, the Luc gene was included in the vector, and fluorescence was measured in the liver.

Each of the PBS, AAV9, T4-AAV9 of Sample 1, and T4-AAV9 of Sample 2 was administered into the cerebrospinal fluid of mice in the same manner as the method described in Example 6.1.

Livers were fixed with 4% paraformaldehyde and then stored in 30% sucrose until used for experiments. Thereafter, an experiment was performed using an immunohistochemical staining experimental method, and tissue samples (liver section) were reacted with the GFP antibody O/N at 4° C.

Thereafter, the tissue sample (liver section) was reacted at 25° C. for 1 hour using an appropriate fluorescent antibody (Alexa Fluor 488) or fluorescent primary antibody (Fluoro Myelin-Red/Alexa Fluor 647-tubulin beta 3).

After the reaction, the tissue sample was analyzed with a Carl Zeiss confocal microscope, and immunofluorescence images of the tissue sample were obtained.

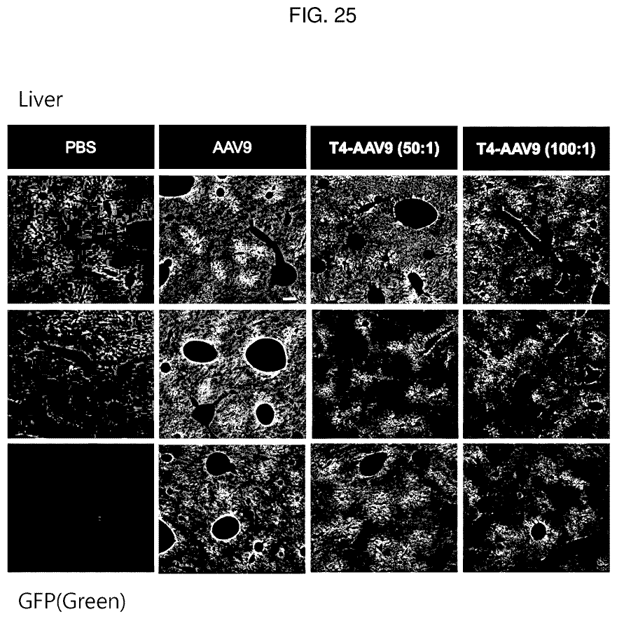

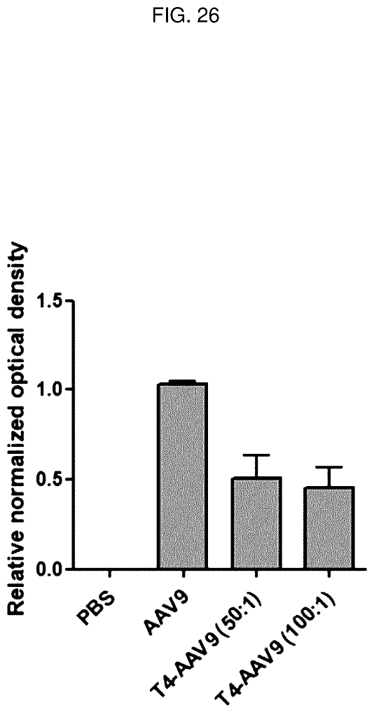

The experimental results are shown in FIGS. 25 and 26. In FIGS. 25 and 26, the group denoted as T4-AAV9 (50:1) means a group treated with T4-AAV9 of Sample 1, and the group denoted as T4-AAV9 (100:1) means a group treated with T4-AAV9 of Sample 2.

In FIGS. 25 and 26, the intensity of fluorescence generated from GFP in the liver was observed for each condition. It was confirmed that the fluorescence observed in the liver in the experimental groups administered Sample 1 and Sample 2 was lower than the fluorescence observed in the group administered the AAV9 sample, which is a comparison group. This indicates that when T4-AAV9 is administered into cerebrospinal fluid, fewer vectors leak from the cerebrospinal fluid and accumulate in the liver than when AAV9 is administered.

Therefore, when using a vector in which the TTR ligands are conjugated, an active substance can be more efficiently delivered to the central nervous system.

What is claimed is:

1. A vector for delivering an active substance in which one or more TTR ligands are conjugated to a surface of an adeno-associated virus, represented by formula 1: 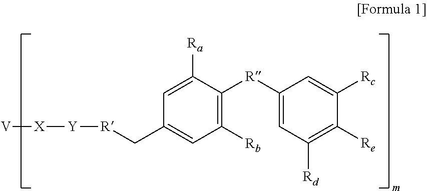
wherein,
V—X— is an adeno-associated virus moiety derived from the adeno-associated virus,
X is —NH— or —S—, wherein X is derived from an amino acid residue present on a capsid of the adeno-associated virus,
Y is -(L)n-, —C(═O)-(L)n-, —C(═NH₂⁺)-(L)n-, —S-(L)n-, or 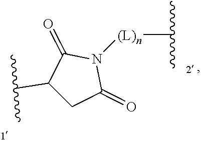
wherein 1′ is linked to X and 2′ is linked to R′,
wherein L is a linker moiety, and wherein L is substituted or unsubstituted C₁₋₂₀ alkylene, substituted or unsubstituted C₂₋₂₀ alkenylene, substituted or unsubstituted C₂₋₂₀ alkynylene, substituted or unsubstituted C₁₋₂₀ heteroalkylene, substituted or unsubstituted C₂₋₂₀ heteroalkenylene, or substituted or unsubstituted C₂₋₂₀ heteroalkynylene,
wherein the heteroalkylene, heteroalkenylene, or heteroalkynylene comprises one or more heteroatoms selected from the group consisting of O, N, and S,
wherein the substitution refers to being substituted with one or more non-hydrogen substituents, wherein the non-hydrogen substituent is any one selected from —R¹, —C(R¹)₃, —OR¹, —SR¹, —N(R¹)₂, —N═C═O, —NCS, —NO, —NO₂, ═N₂, —N₃, —NHC(═O)R¹, ═O, ═S, —C(═O)R¹, —C(═O)OR¹, —C(═O)NR¹R¹, —S(═O)₂R¹, —OS(═O)₂OR¹, —S(═O)₂NR¹, —S(═O)R¹, alkylene-C(═O)R¹, —C(═S)R¹, alkylene-C(═O)OR¹, —C(═S)OR¹, —C(═O)SR¹, —C(═S)SR¹, —C(═O)NR¹R¹, alkylene-C(═O)NR¹R¹, —C(═S)NR¹R¹, —C(—NR¹)NR¹R¹, and
wherein R¹ is H, halogen, C₁₋₆ alkyl, C₂₋₆ alkene, C₂₋₆ alkyne, C₁₋₆ heteroalkyl, C₆₋₁₂ aryl, C₇₋₁₂ arylalkyl or C₃₋₁₀ cycloalkyl, C₃₋₁₀ cycloalkene, C₃₋₁₀ cycloalkyn, or C₃₋₁₀ heterocycloalkyl,
n is 0 or 1,
R′ is —C(═O)—, C₁₋₃ alkylene, 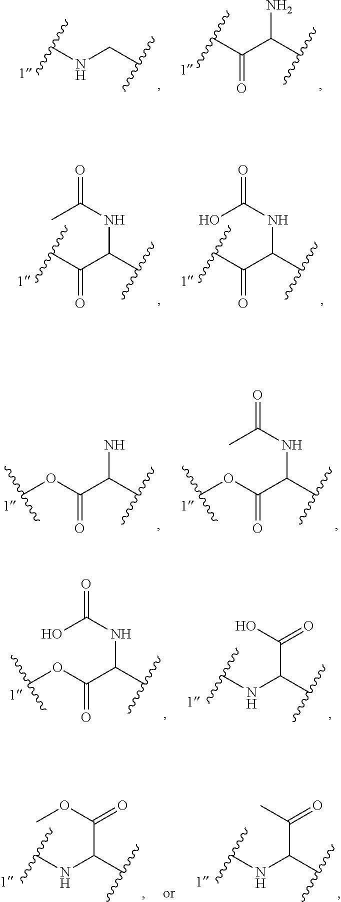
wherein 1″ is linked to Y,
Ra is H, C₁₋₃ alkyl, or I,
Rb is H, C₁₋₃ alkyl, or I,
R″ is —O—, —S—, —NH—, or —CH₂—,
Rc is H, C₁₋₃ alkyl, or I,
Rd is H, C₁₋₃ alkyl, or I,
Re is OH, and
m is an integer 2 or more.

2. The vector for delivering the active substance of claim 1, wherein X is —NH—, wherein X is derived from an amine residue of a lysine present on the capsid of the adeno-associated virus.

3. The vector for delivering the active substance of claim 1, wherein X is —S—, wherein X is derived from a thiol residue of a cysteine present on the capsid of the adeno-associated virus.

4. The vector for delivering the active substance of claim 1, wherein Ra is I; Rb is H or I; R″ is —O—; Rc is H or I; and Rd is H or I.

5. The vector for delivering the active substance of claim 1, wherein the vector in which one or more TTR ligands are conjugated to the surface of the adeno-associated virus is represented by formula 1-2: 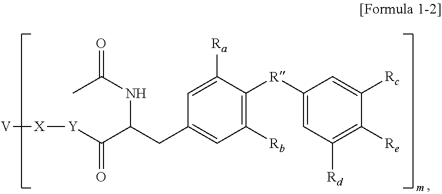
wherein,
V—X— is the adeno-associated virus moiety derived from the adeno-associated virus,
X is —NH— or —S—, wherein X is derived from the amino acid residue present on the capsid of the adeno-associated virus,
Y is -(L)n-, —C(═O)-(L)n-, —C(═NH₂⁺)-(L)n-, —S-(L)n-, or 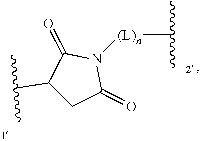
and wherein 1′ is linked to X and 2′ is linked to R′,
wherein L is the linker moiety, and wherein L is unsubstituted C₁₋₁₀ alkylene, unsubstituted C₂₋₁₀ alkenylene, unsubstituted C₂₋₁₀ alkynylene, or unsubstituted C₁₋₁₀ heteroalkylene, and
wherein the heteroalkylene comprises one or more heteroatoms selected from the group consisting of O, N, and S,
n is 0 or 1,
Ra is H, C₁₋₃ alkyl, or I,
Rb is H, C₁₋₃ alkyl, or I,
R″ is —O—, —S—, —NH—, or —CH₂—,
Rc is H, C₁₋₃ alkyl, or I,
Rd is H, C₁₋₃ alkyl, or I,
Re is OH, and
m is an integer 2 or more.

6. The vector for delivering an active substance of claim 5, wherein Ra is I; Rb is H or I; R″ is —O—; Rc is H or I; and Rd is H or I.

7. The vector for delivering an active substance of claim 6, wherein Y is -(L)n-.

8. The vector for delivering the active substance of claim 1, wherein the vector in which one or more TTR ligands are conjugated to the surface of the adeno-associated virus is represented by formula 1-3: 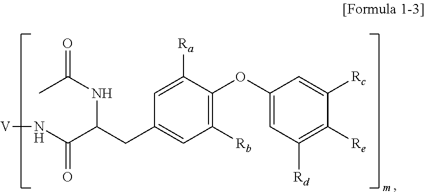
wherein,
V—NH— is the adeno-associated virus moiety derived from the adeno-associated virus,
wherein, in the adeno-associated virus moiety, —NH— is derived from an amine residue of a lysine present on the capsid of the adeno-associated virus,
Ra is H, methyl, or I,
Rb is H, methyl, or I,
Rc is H, C₁₋₃ alkyl, or I,
Rd is H, C₁₋₃ alkyl, or I,
Re is OH, and
m is an integer 2 or more.

9. The vector for delivering the active agent of claim 8, wherein Ra is H or I; Rb is H or I; Rc is H or I; and Rd is H or I.

10. The vector for delivering the active substance of claim 1, wherein the vector in which one or more TTR ligands are conjugated to the surface of the adeno-associated virus is represented by formula 1-4: 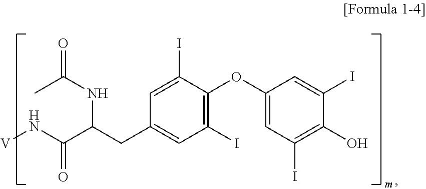
wherein,
V—NH— is the adeno-associated virus moiety derived from the adeno-associated virus,
wherein, in the adeno-associated virus moiety, —NH— is derived from an amine residue of a lysine located in the capsid of the adeno-associated virus, and
m is an integer 2 or more.

11. The vector for delivering the active substance of claim 1, wherein the vector in which one or more TTR ligands are conjugated to the surface of the adeno-associated virus is represented by formula 1-5: 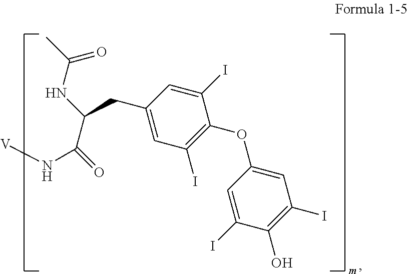
wherein,
V—NH— is the adeno-associated virus moiety derived from the adeno-associated virus,
wherein, in the adeno-associated virus moiety, —NH— is derived from an amine residue of a lysine located in the capsid of the adeno-associated virus, and
m is an integer 2 or more.

12. The vector for delivering the active substance of claim 1, wherein the vector comprises one or more active substances, wherein the active substance is a transgene.

13. The vector for delivering the active substance of claim 12, wherein the active substance is comprised in a virus particle of the adeno-associated virus moiety.

14. The vector for delivering the active substance of claim 12, wherein the transgene is a gene related to a treatment of neurological disorder.

15. The vector for delivering the active substance of claim 14, wherein the neurological disorder is central nervous system disorder or peripheral nervous system disorder.

16. The vector for delivering the active substance of claim 14, wherein the gene related to the treatment of neurological disorder is any one selected from MECP2, SCN1A, NF2, SNCA, LRRK2, APP, Tau, Nav1.7, C9rof72, SOD1, DYRK1A, IT15, HTT, HEXA, RA11, PRGN, UBE3A, ABCA4, RP1, PAX6, USH2A, NRP1, SMN1, SMN2, GAA, PMP22, TTR, APOEε4, APOEε3, APOEε2, MAPT, GRN, AADC, GBA1, ASPA, CLN2, GLB1, SGSH, NAGLU, IDS, NPC1, SCA1(ATXN1), SCA3(ATXN3), SCA7(ATXN7), TARDBP(TDP-43), FRDA(FXN), SCN9A, SCN10A, and GAN.

17. A composition for delivering an active substance to central nervous system or peripheral nervous system, comprising a vector for delivering an active substance of claim 12.
